(19) United States
(12) Patent Application Publication
(10) Pub. No.: US 2026/0007579 A1
(43) Pub. Date: Jan. 8, 2026

(54) METHODS AND DEVICES FOR HYDROLYZING FATS

(71) Applicant: Alcresta Therapeutics, Inc., Waltham, MA (US)

(72) Inventors: Greta L. Loring, Wakefield, MA (US); Craig Fairchild, Acton, MA (US); Stephen Davia, Dorchester, MA (US)

(73) Assignee: Alcresta Therapeutics, Inc., Waltham, MA (US)

(21) Appl. No.: 19/294,113

(22) Filed: Aug. 7, 2025

Related U.S. Application Data
(63) Continuation of application No. 19/260,039, filed on Jul. 3, 2025.
(60) Provisional application No. 63/667,960, filed on Jul. 5, 2024.
(60) Provisional application No. 63/713,331, filed on Oct. 29, 2024.

(51) Int. Cl. C12P 7/6418; A23L 33/00; A61J 15/00; A61K 9/00; A61K 31/20; A61K 31/23; A61M 5/145

(52) U.S. Cl. CPC C12P 7/6418; A23L 33/40; A61J 15/0011; A61J 15/0076; A61K 9/0053; A61K 31/20; A61K 31/23; A61M 5/14546

(57) ABSTRACT

A method of preparing a nutritional composition, the method comprising connecting a first end of a hydrolysis device to a syringe and connecting a second end of the hydrolysis device to an enteral straw, wherein the hydrolysis device comprises lipase; drawing a nutritional composition into the syringe via the enteral straw, wherein the nutritional composition runs through the hydrolysis device in a first direction and the nutritional composition includes triglycerides; replacing the enteral straw with a feeding tube; and expelling the nutritional composition from the syringe through the feeding tube, wherein the nutritional composition runs through the hydrolysis device in a second direction, resulting in a hydrolyzed nutritional composition.

### CROSS-REFERENCE TO RELATED APPLICATIONS

This application is a continuation of and claims the benefit of priority from U.S. patent application Ser. No. 19/260,039, filed Jul. 3, 2025, which claims the benefit of priority from U.S. Provisional Application Nos. 63/713,331, filed Oct. 29, 2024, and 63/667,960, filed Jul. 5, 2024, the entireties of which are incorporated herein by reference.

### TECHNICAL FIELD

Embodiments of the present disclosure are directed to devices and methods for preparing and/or supplementing a nutritional composition.

### BACKGROUND

Commercially available nutritional formulas, e.g., enteral formulas for adults, children, and infants, may have variable contents, such as fat emulsion stabilizers, soluble and insoluble fiber, and protein components. These differences may have a significant impact on how efficiently fat is hydrolyzed ex vivo, i.e., outside of the living body. Some formulas are particularly challenging to hydrolyze, especially at faster flow rates used in bolus feeding, e.g., flow rates of up to about 400 ml/hour. Systems and methods to more efficiently and effectively hydrolyze nutritional formulas are described herein.

### SUMMARY OF THE EMBODIMENTS

The present disclosure describes a method of preparing a nutritional composition, the method comprising connecting a first end of a hydrolysis device to a syringe and connecting a second end of the hydrolysis device to an enteral straw, wherein the hydrolysis device comprises lipase; drawing a nutritional composition into the syringe via the enteral straw, wherein the nutritional composition runs through the hydrolysis device in a first direction and the nutritional composition includes triglycerides; replacing the enteral straw with a feeding tube; and expelling the nutritional composition from the syringe through the feeding tube, wherein the nutritional composition runs through the hydrolysis device in a second direction, resulting in a hydrolyzed nutritional composition.

Various embodiments of the method may include one or more of the following features. The lipase may be immobilized such that when the hydrolyzed nutritional composition exits the hydrolysis device, the immobilized lipase remains in the hydrolysis device. The lipase may be immobilized to a structure within the container. The structure may comprise balls or beads within the container. The method may further comprise connecting the hydrolysis device to a syringe pump. Drawing the nutritional composition into the syringe via the enteral straw in the first direction may allow the lipase to at least partially hydrolyze the triglycerides. The method may further comprise delivering the nutritional composition to a subject for digestion. The nutritional composition may be drawn into the syringe via the enteral straw in the first direction for a time duration ranging from about 1 second to about 5 minutes. The lipase may be selected from a group consisting of Chromobacterium viscosum lipase, Pseudomonas fluorescens lipase, and Rhizopus oryzae lipase. The method may further comprise delivering the nutritional composition to a subject having at least one of Alzheimer's disease, bipolar disorder, depression, sepsis, acute respiratory stress, cancer, cardiovascular disease, stroke, Parkinson's disease, schizophrenia, diabetes, multiple sclerosis, malnutrition, impaired GI function, rheumatoid arthritis, systemic lupus erythematosus, inflammatory bowel disease, short bowel syndrome, irritable bowel disorder, Systemic Inflammatory Response Syndrome, celiac disease, Crohn's disease, Cystic Fibrosis, Zollinger-Ellison syndrome, hypertriglyceridemia, neoplasms, hemochromatosis, primary sclerosing cholangitis, primary biliary cirrhosis, diarrhea, Shwachman's syndrome, trypsinogen deficiency, enterokinase deficiency, chylothorax, isolated deficiency of lipase, necrotizing enterocolitis, premature birth, pancreatic insufficiency, pancreatitis, malabsorption, compromised pancreatic output, a reduced ability to hydrolyze triglycerides or esters, or a reduced ability to absorb triglycerides or esters. The method may further comprise providing the nutritional composition to a subject that is an infant.

The present disclosure also describes a method of preparing a nutritional composition for ingestion by a subject, the method comprising running a nutritional composition through a hydrolysis device for a first pass, wherein the hydrolysis device includes a first end fluidly coupled to a syringe, a second end fluidly coupled to an enteral straw, and immobilized lipase, wherein the first pass of the nutritional composition flows from the enteral straw, through the hydrolysis device, and to the syringe; and running the nutritional composition through the hydrolysis device for a second pass, wherein the second pass of the nutritional composition flows from the syringe, through the hydrolysis device, and through the second end of the hydrolysis device.

Various embodiments of the method may include one or more of the following features. The first pass of the nutritional composition may last for a time duration ranging from about 1 second to about 5 minutes. The method may further comprise replacing the enteral straw with a feeding tube, wherein during the second pass the nutritional composition flows through the second end of the hydrolysis device and into the feeding tube. The method may further comprise delivering the nutritional composition to a subject for digestion. The method may further comprise connecting the hydrolysis device to a syringe pump. The lipase may be immobilized such that when the hydrolyzed nutritional composition exits the hydrolysis device, the immobilized lipase remains in the hydrolysis device. The nutritional composition may include triglycerides and wherein the first pass of the nutritional composition and the second pass of the nutritional composition may allow the lipase to at least partially hydrolyze the triglycerides. The method may further comprise delivering the nutritional composition to a subject having at least one of Alzheimer's disease, bipolar disorder, depression, sepsis, acute respiratory stress, cancer, cardiovascular disease, stroke, Parkinson's disease, schizophrenia, diabetes, multiple sclerosis, malnutrition, impaired GI function, rheumatoid arthritis, systemic lupus erythematosus, inflammatory bowel disease, short bowel syndrome, irritable bowel disorder, Systemic Inflammatory Response Syndrome, celiac disease, Crohn's disease, Cystic Fibrosis, Zollinger-Ellison syndrome, hypertriglyceridemia, neoplasms, hemochromatosis, primary sclerosing cholangitis, primary biliary cirrhosis, diarrhea, Shwachman's syndrome, trypsinogen deficiency, enterokinase deficiency, chylothorax, isolated deficiency of lipase, necrotizing enterocolitis, premature birth, pancreatic insufficiency, pancreatitis, malabsorption, compromised pancreatic output, a reduced ability to hydrolyze triglycerides or esters, or a reduced ability to absorb triglycerides or esters. The method may further comprise providing the nutritional composition to a subject that is an infant.

The present disclosure also describes a method of preparing a nutritional composition, the method comprising connecting a first end of a hydrolysis device to a syringe, wherein the hydrolysis device comprises lipase, drawing a nutritional composition into the syringe so that the nutritional composition runs through the hydrolysis device in a first direction, and wherein the nutritional composition includes triglycerides; and expelling the nutritional composition from the syringe, wherein the nutritional composition runs through the hydrolysis device in a second direction, opposite the first direction, wherein the drawing and the expelling steps result in a hydrolyzed nutritional composition in which at least some of the triglycerides are hydrolyzed into free fatty acids and monoglycerides. The nutritional composition may run through the hydrolysis device in the first direction for a time duration ranging from about 1 second to about 5 minutes.

### BRIEF DESCRIPTION OF THE DRAWINGS

The accompanying drawings, which are incorporated into and constitute a part of this specification, illustrate various exemplary embodiments and, together with the description, serve to explain the principles of the disclosed embodiments. The drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

There are many embodiments described and illustrated herein. The described embodiments are neither limited to any single aspect nor embodiment thereof, nor to any combinations and/or permutations of such aspects and/or embodiments. Moreover, each of the aspects of the described embodiments may be employed alone or in combination with one or more of the other aspects of the described inventions and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

FIG. 1 illustrates a device and system for preparing a nutritional composition.

FIGS. 2A, 2B, and 2C illustrate systems and steps for preparing a nutritional composition, according to certain embodiments of the present disclosure.

FIGS. 3A, 3B, and 3C illustrate systems and steps for preparing a nutritional composition, according to certain embodiments of the present disclosure.

FIGS. 4A, 4B, and 4C illustrate a device and its components for preparing a nutritional composition, according to certain embodiments of the present disclosure.

FIG. 5A is a flow chart illustrating a method for preparing a nutritional composition.

FIG. 5B is a flow chart illustrating a method for preparing a nutritional composition.

FIG. 5C is a flow chart illustrating a method for preparing a nutritional composition.

FIG. 5D is a flow chart illustrating a method for preparing a nutritional composition.

FIG. 6 schematically depicts the study design and procedures for the specimens of Groups 1-5 described in Example 6.

FIG. 7A graphically compares the survival rates of the specimen after the procedures described in Example 6.

FIG. 7B graphically compares the body weight of the specimen after the procedures described in Example 6.

FIG. 8A graphically compares the clinical sickness score of the specimen after the procedures described in Example 6.

FIG. 8B graphically compares the macroscopic gut score of the specimen after the procedures described in Example 6.

FIG. 8C graphically compares the histologic severity of the specimen after the procedures described in Example 6.

FIG. 9A shows the intestinal appearance of an exemplary specimen of Group 1 after the procedures described in Example 6.

FIG. 9B shows the intestinal appearance of an exemplary specimen of Group 2 after the procedures described in Example 6.

FIG. 9C shows the intestinal appearance of an exemplary specimen of Group 3 after the procedures described in Example 6.

FIG. 9D shows the intestinal appearance of an exemplary specimen of Group 4 after the procedures described in Example 6.

FIG. 9E shows the intestinal appearance of an exemplary specimen of Group 5 after the procedures described in Example 6.

FIG. 10A depicts a representative histological image of the terminal ileum specimens of Group 1 after the procedures described in Example 6.

FIG. 10B depicts a representative histological image of the terminal ileum specimens of Group 2 after the procedures described in Example 6.

FIG. 10C depicts a representative histological image of the terminal ileum specimens of Group 3 after the procedures described in Example 6.

FIG. 10D depicts a representative histological image of the terminal ileum specimens of Group 4 after the procedures described in Example 6.

FIG. 10E depicts a representative histological image of the terminal ileum specimens of Group 5 after the procedures described in Example 6.

FIG. 11A depicts a representative histological image of the small intestine specimens of Group 1 after the procedures described in Example 6.

FIG. 11B depicts a representative histological image of the small intestine specimens of Group 2 after the procedures described in Example 6.

FIG. 11C depicts a representative histological image of the small intestine specimens of Group 3 after the procedures described in Example 6.

FIG. 11D depicts a representative histological image of the small intestine specimens of Group 4 after the procedures described in Example 6.

FIG. 11E depicts a representative histological image of the small intestine specimens of Group 5 after the procedures described in Example 6.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items. Further, as used herein, the terms “about,” “substantially,” and “approximately” generally mean+/−10% of the indicated value. Additionally, the term “between” as used herein includes the end points of a stated range, unless the end points are explicitly excluded. As used herein, the term “nutritional composition” refers to both nutritional formulas, as well as milk, which may be human or animal donor milk or breast milk.

### DETAILED DESCRIPTION

Fatty acids, e.g., short, medium, and long-chain fatty acids, are critical to human health and development. Long-chain fatty acids that are consumed in the diet are primarily in the form of triglycerides (TGs), in which three long-chain fatty acids are bound to a glycerol molecule via ester linkages. Absorption of triglycerides first requires the enzymatic action of lipases, (e.g. pancreatic lipase), which digest triglycerides through hydrolysis, breaking them down into monoglycerides and further into free fatty acids. Once available, these monoglycerides and free fatty acids are absorbed by endothelial cells in the small intestine, where they undergo reesterification, followed by transport to the liver and ultimately to tissues in the body for various physiological purposes. Kasper et al., Harrison's Principles of Internal Medicine 16^{th} Ed. (2004). While medium chain triglycerides can be absorbed across the intestinal lumen, long-chain triglycerides cannot, therefore, pancreatic lipase is essential for proper long-chain fatty acid hydrolysis and absorption. Jensen et al., Am. J. Clin. Nutr. 43:745-751 (1986). However, some people are unable to adequately breakdown long-chain triglycerides, e.g., patients suffering from compromised pancreatic output, malabsorption or pancreatic insufficiency, and as a result, may suffer from absorption of fatty acids that is inadequate to maintain health.

Commercially available lipase supplements may be added to the diet to improve hydrolysis of triglycerides. However, for a number of reasons, lipase supplements will not necessarily solve the problem of poor fatty acid absorption in all people suffering from reduced ability to break down triglycerides or otherwise in need of receiving elemental fatty acids. For example, most commercial lipase supplements are made from animal pancreatic lipase, which is known to have significantly reduced stability below pH 7. See, e.g., US2010/0239559; Kasper et al., Harrison's Principles of Internal Medicine 16^{th} Ed. (2004). By the time such lipases pass through the stomach, significant amounts are likely to have been inactivated. Further, not all lipases work to the same degree for hydrolysis of a given fatty acid, e.g., a long-chain fatty acid, indicating lipase specificity is an important consideration. Jensen et al., Lipids 18(3): 239-252 (1983). And in some populations with pancreatic insufficiency, nutritional formulas are tightly regulated, such as infants, pre-term infants, or patients in intensive care units. For these controlled populations, it may not be desirable or feasible to supplement already-approved formulas with additional ingredients. Moreover, although many formulas may contain medium-chain triglycerides, there is a distinct medical benefit to dietary intake of long-chain fatty acids. There is a need for improved methods of enhancing hydrolysis of triglycerides, e.g., long-chain triglycerides.

Current systems and methods temporarily expose a nutritional composition, such as nutritional formula, e.g., a medical nutritional formula or an infant formula, comprising fats such as triglycerides and/or fatty acid esters, to lipase in order to hydrolyze the fats prior to consumption. Exposing such nutritional compositions to lipase prior to ingestion allows for the breakdown of fats into free fatty acids and monoglycerides, without requiring ingestion of exogenous lipase. Such hydrolysis devices may have an inlet for receiving nutritional composition, a chamber containing immobilized lipase, and an outlet for allowing nutritional composition to exit the device. Nutritional composition may enter through the inlet, then may be exposed to the lipase within the chamber, where fats, e.g., triglycerides, within the formula may be at least partially hydrolyzed, and then the hydrolyzed nutritional composition may exit the chamber. Exemplary hydrolysis devices are discussed further in detail in U.S. Pat. Nos. 10,258,590 and 9,668,942, incorporated herein by reference in their entireties. An exemplary hydrolysis device is the RELIZORB® device, which is an enteral device containing immobilized lipase, sold by Alcresta Therapeutics. After the nutritional composition has completely passed through the hydrolysis device and has been at least partially hydrolyzed by the immobilized lipase, the nutritional composition may contain more monoglycerides and/or free fatty acids, but may contain no significant amount of exogenous lipase. In some embodiments, a hydrolyzed nutritional composition may not comprise added lipase. A nutritional composition that does not comprise added lipase refers to a formula in which lipase is not detectable or is present only at very low levels, due, e.g., to leaching of immobilized lipase from a solid support into the formula. In some embodiments, a nutritional composition comprises no more than 0.02% (w/w) lipase, no more than 0.01% (w/w) lipase, no more than 0.005% (w/w) lipase, no more than 0.002% (w/w) lipase, no more than 0.001% (w/w) lipase, no more than 0.0005% (w/w) lipase, no more than 0.0002% (w/w) lipase, or no more than 0.0001% (w/w) lipase. In some embodiments, a nutritional composition comprises less than 0.02% (w/w) lipase, less than 0.01% (w/w) lipase, less than 0.005% (w/w) lipase, less than 0.002% (w/w) lipase, less than 0.001% (w/w) lipase, less than 0.0005% (w/w) lipase, less than 0.0002% (w/w) lipase, or less than 0.0001% (w/w) lipase.

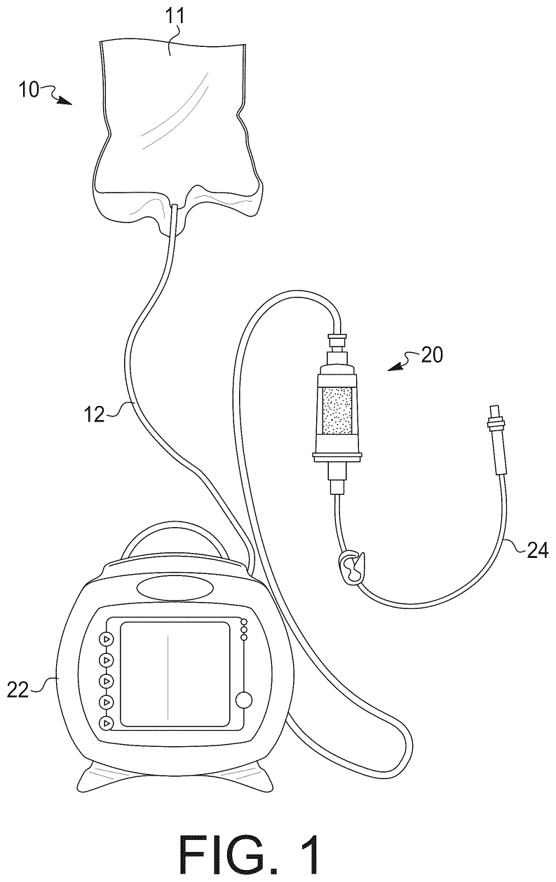

During use, enteral feeding hydrolysis devices for hydrolyzing nutritional compositions, such as the RELIZORB® device, may be utilized in a larger enteral system, as shown in FIG. 1. The system of FIG. 1 shows an enteral feeding system 10 for feeding a nutritional composition 11 to a subject via a feeding tube. System 10 includes a hydrolysis device 20 connected in line with feeding tubes 12 and 24, a source of nutritional composition 11 (such as a feeding bag, as shown), and a pump 22 to control the flow of the nutritional composition from the source of nutritional composition 11, through feeding tube 12, through the hydrolysis device 20, and through feeding tube 24 and to the subject. An inlet of hydrolysis device 20 is connected to feeding tube 12 to receive nutritional composition within the device, and an outlet of hydrolysis device 20 is connected to feeding tube 24, to allow hydrolyzed nutritional composition to be delivered to the subject. Feeding tube 24 may be a nasogastric, a nasoduodenal, a nasojejunal, a gastrostomy, a gastrojejunostomy, a jejunostomy, a PEG tube, or a transjejunal feeding tube to feed nutritional composition to the GI tract of a subject through, for example, the nose, mouth, stomach, or abdomen.

Systems such as the one shown in FIG. 1 are the traditional method for delivering hydrolyzed nutritional compositions to a patient using a hydrolysis device 20. They have been used successfully for years, and such systems allow for a one-way flow of nutritional composition from a source of nutritional composition 11 and to a subject. However, use of such systems over the years have led to the realization of some drawbacks that were not initially known.

For example, commercially available nutritional formulas, including, e.g., enteral formula for adults or children and infant formulas, vary widely in their ingredients. Different formulas have different amounts of fat emulsion stabilizers, soluble and insoluble fiber and protein components, and thus different viscosities. These differences between formulas may significantly impact how efficiently fats such as triglycerides are hydrolyzed by a hydrolysis device. As a result, hydrolysis devices may yield unacceptably low hydrolysis rates when used with certain formulas. It may be difficult, however, to switch a subject to a different formula that works better with a hydrolysis device, because subjects may respond differently to different formulas, and an individual subject may thus be unwilling or unable to switch to a different formula once he or she has found one that works for them. Further, it may not be possible to slow the rate at which nutritional formula is passed through the hydrolysis device, for example, by slowing a flow rate of pump 22, because then feeding times may be too long, and slower rates may be incompatible with bolus delivery. Accordingly, traditional delivery methods and systems may be insufficient for providing sufficiently hydrolyzed nutritional formulas to a subject.

Another issue with current systems and methods is that current systems include many components, such as longer feeding tubes 12, 24, as well as hydrolysis device 20, through which the nutritional composition must flow. Portions of the nutritional composition, such as the nutrients and fats that the subject needs, may be thicker or sticky and may adhere to the walls of feeding tubes 12, 24, resulting in a loss of nutrients for the patient. Additionally, the lipase in hydrolysis device 20 may be immobilized to structures, such as beads, within the device. In current devices in which the lipase is immobilized to beads, the immobilized lipase beads may form a bead bed that the nutritional composition flows through. Portions of the nutritional composition and/or components of the nutritional composition may adhere to parts of the device and/or the immobilized lipase bead beds resulting in a loss of nutrients that are provided to the subject. As the nutritional composition flows through the hydrolysis device, a portion of the fats and nutrients in the nutritional composition may get stuck within the bead bed of immobilized lipase, again resulting in a loss of valuable nutrients and fats within the device that ultimately do not make it to the subject.

The immobilized lipase beads utilized in current systems and methods may be formed by various methods, for example, adsorption, ionic binding, non-covalent binding, cross-linking, encapsulation, and entrapment onto hydrophobic or hydrophilic polymeric and inorganic matrices. Such methods may include drying the lipase onto the beads. As such, the resulting immobilized lipase beads may be extremely dry. Further, the beads utilized in current systems and methods may not expand in water-based liquids, e.g., nutritional compositions, as much as compared to the same beads when placed in an organic solvent. As the nutritional composition flows through hydrolysis devices utilized in current systems and methods, the beads' dryness and decreased ability to expand in water-based liquids, e.g., nutritional compositions, may result in a decreased amount of fats that are hydrolyzed.

Embodiments of the present disclosure may address one or more of these problems by introducing a novel method of processing and delivering a nutritional composition using a hydrolysis device such as the RELIZORB® device. Embodiments of the disclosure are drawn to a redesigned system and method of delivery that allows for passing the nutritional composition through the hydrolysis device multiple times in order to increase hydrolysis of fats within the nutritional composition. Traditional systems, such as the one shown in FIG. 1, would not have been capable of multiple passes through the hydrolysis device, because the traditional enteral pump system only allows for one-way flow of a nutritional composition from a source, through the hydrolysis device a single time, and to a subject. Further, since the problems described above with the current hydrolysis devices and enteral systems were not recognized previously, there would have been no reason to move away from the systems described in FIG. 1.

Throughout this disclosure, devices and methods will be referred to for use in preparing or delivering nutritional composition. As used herein, the term “nutritional formula” refers to complex mixtures containing, for example, proteins, carbohydrates, fats, water, minerals, and/or vitamins, which may include liquid foods that are specially formulated and processed; liquids used for the partial or exclusive feeding of a subject by means of oral intake or feeding by tube; liquids used for the dietary management of a subject who, because of therapeutic or medical need, has limited or impaired capacity to ingest, digest, absorb, or metabolize ordinary foodstuffs or certain nutrients; liquids that meet medically determined nutrient requirements; and liquids designed to deliver to a subject nutrients that cannot be provided to the subject via dietary management and modification of the normal diet alone. The term nutritional formula may also include formulas intended for the specific dietary management of a disease, condition, or state (such as age) for which distinctive nutritional requirements, based on recognized scientific principles, are established by medical evaluation, or may include liquid foods used as part of an overall diet to manage the symptoms or reduce the risk of a disease or condition. In some embodiments, a nutritional formula may be delivered to the subject under medical supervision, may be intended only for a subject receiving active and ongoing medical supervision, or may be delivered to the subject for home use, either when supervised or unsupervised.

Exemplary nutritional formulas may be packaged as a dry powder or oil and then mixed with a solvent to form a solution. In other embodiments, nutritional formulas may be packaged as a liquid nutritional formula, beverage, or drink. In some embodiments, nutritional formulas may be commercially available, or may be prepared, e.g., by an individual or a healthcare professional, before feeding. Nutritional formulas may be an infant and/or toddler formula as a complete or partial substitute for human milk, or may be designed to supplement or completely replace the diet of an adult or elderly person. In some embodiments, nutritional formulas may be a commercially available or a custom-developed formula combined with a commercially available or a custom-developed supplement or fortifier, which may supply additional nutrients including, but not limited to, one or more of long-chain triglycerides, medium-chain triglycerides, vitamins, minerals, or proteins. In some embodiments, nutritional formula may be conditioned to make fats contained in it more accessible for hydrolysis. Exemplary conditioning may include one or more of sonication, fat droplet disruption, or emulsification, e.g., by physical or chemical means (e.g. by exposure to a surfactant, surfactant-like substance, or protease). In some embodiments, nutritional formulas may be prescribed for a subject in need of additional long-chain polyunsaturated fatty acids (LC-PUFAs), such as DHA, EPA, and/or AA, a subject having conditions such as maldigestion and malabsorption of lipids, reduced caloric intake, significant weight loss, LC-PUFA deficiencies, and/or a subject having diseases, including cystic fibrosis (CF), chronic pancreatitis (CP), necrotizing enterocolitis (NEC), surgery, cancer, liver abnormalities, gastrointestinal dysfunction, and developmental immaturity. In some embodiments, the subject may have exocrine pancreatic insufficiency (EPI) with reduced ability to hydrolyze long-chain triglycerides. In some embodiments, nutritional formulas may include at least one medicament prescribed for the subject in need of the medicament and/or nutritional formula, or nutritional formula may itself be a prescribed medicament.

Nutritional formulas may include at least one fat in triglyceride form, such as medium or long chain triglycerides. In some embodiments, nutritional formulas may further include at least one nutrient selected from water, maltodextrin, protein, hydrolyzed protein, amino acids, peptides, medium chain triglycerides, diglycerides, monoglycerides, cornstarch, fish oil, soybean oil, rapeseed oil, cottonseed oil, sunflower oil, olive oil (oils may or may not be refined), soluble fiber, lecithin, magnesium chloride, sodium ascorbate, guar gum, calcium phosphate, salt, choline chloride, phosphoric acid, calcium citrate, sodium phosphate, taurine, magnesium oxide, zinc sulfate, potassium chloride, niacinamide, ferrous sulfate, calcium pantothenate, manganese sulfate, pyridoxine hydrochloride, copper sulfate, thiamine mononitrate, beta-carotene, riboflavin, vitamin a palmitate, folic acid, biotin, sodium selenate, chromium chloride, potassium iodide, sodium molybdate, soluble fiber, fructooligosaccharide, probiotic, citric acid, vitamin A, vitamin D, vitamin E, vitamin B₁, vitamin B₂, vitamin B₃, vitamin B₅, vitamin B₆, vitamin B₇, vitamin B₉, and vitamin B₁₂.

In some embodiments, a nutritional composition is exposed to lipase prior to ingestion. In some embodiments, this exposure allows pre-hydrolysis of at least some lipids in the nutritional composition. Thus, in some embodiments, a nutritional composition is an “as-fed” formula or milk, i.e., the liquid formula or milk as composed just prior to ingestion by the subject, which differs in composition from the formula as sold by the manufacturer or un-hydrolyzed milk. The term “nutritional composition” does not encompass compositions existing within the body of a subject after ingestion.

In some embodiments, the approximate serving size of a nutritional formula, after hydrolyzation, may be about 5-110 mL for premature infant formula, 90-150 mL (e.g., 100 mL) for term infant formula, 230-500 mL (e.g., 235-250 mL) for enteral feeds, and 230-250 mL for child formulas and adult formulas. In some embodiments, a serving of a hydrolyzed nutritional formula may contain 50-100 mg of free fatty acids and/or monoglycerides. Serving sizes may be similar for milk. In some embodiments, a serving of a nutritional composition may contain 100-200 mg of free fatty acids and/or monoglycerides. In some embodiments, a serving of a nutritional composition may contain 200-300 mg of free fatty acids and/or monoglycerides. In some embodiments, a serving of a nutritional composition may contain 250-500 mg of free fatty acids and/or monoglycerides. In some embodiments, a serving of a nutritional composition may contain 500-1000 mg of free fatty acids and/or monoglycerides. In some embodiments, a serving of a nutritional composition may contain 1-2 grams of free fatty acids and/or monoglycerides. In some embodiments, a serving of a nutritional composition may contain 2-3 grams of free fatty acids and/or monoglycerides, 3-5 grams of free fatty acids and/or monoglycerides, 3-7 grams of free fatty acids and/or monoglycerides, or higher.

### Lipase

Pancreatic insufficiency and other conditions associated with reduced ability to hydrolyze triglycerides or fatty acid esters are currently treated with supplementary digestive enzymes, including pancreatic lipase. However, pancreatic enzymes, and particularly pancreatic lipase present in these supplements, are often sensitive to degradation by gastric acid and pepsin so that only a small fraction of the ingested enzymes reach the duodenum in active form. Ville et al., Digestion 65:73-81 (2001). Unfortunately, many of the acid protective coatings have potential safety concerns for infant populations or immune compromised patients since a significant portion of the delivered weight is the plastic coating. Moreover, although acid protective coatings have helped, some degree of malabsorption persists, causing patients with pancreatic insufficiency to require increasing doses of enzyme supplements. This persistence of fatty acid malabsorption with use of enterally coated enzymes may be due to the fact that the duodenum and upper jejunum in patients with pancreatic insufficiency are often acidic environments, so that the expected rise in pH is not achieved and the protective coating is not properly dissolved to release the enzyme. Graham, New England J. Med. 296(23):1314-1317 (1977). Both of these problems have been addressed by increasing the dose of lipase administered. Unfortunately, high doses of pancreatic enzyme supplements have been found to be associated with fibrosing colonopathy. Thus, some embodiments provide nutritional compositions that comprise higher percentages of monoglycerides and/or free fatty acids without containing added lipase.

Lipases can be obtained from animals, plants, and many natural or genetically engineered microorganisms. Many, if not most, commercially available dietary lipase supplements are derived from animals and are particularly susceptible to degradation by digestive enzymes. A less frequently used alternative is microbial lipase, i.e., lipase produced in bacteria or fungus, such as, e.g., yeast. Microbial lipases retain activity over a wider pH range than animal or plant lipases, thus eliminating the need for enteric coated tablets. However, microbial enzymes tend to be degraded by trypsin in the small intestine, thereby reducing their availability to breakdown triglycerides and esters in the gut. In certain embodiments, the lipase used in the formulas, methods, or devices of the invention are bacterial lipases, fungal lipases, or both.

The specificity and kinetics of individual lipases can vary significantly. Specificity of lipases is controlled by the molecular properties of the enzyme, structure of the substrate and factors affecting binding of the enzyme to the substrate. Types of specificity include substrate specificity, i.e., a given lipase may be more active in breaking down a type of fatty acid than another lipase, and positional specificity, which involves preferential hydrolysis of ester bonds in positions 1 and/or 3 of the glycerol backbone of a triglyceride.

Hydrolysis devices as described herein may include one or more of Chromobacterium viscosum lipase, Pseudomonas fluorescens lipase, Burcholderia cepacia lipase, and/or Rhizopus oryzae lipase. In some embodiments, the lipase may be Chromobacterium viscosum lipase, Pseudomonas fluorescens lipase, or Rhizopus oryzae lipase. In certain embodiments, the lipase may be Rhizopus oryzae lipase.

Reference to the lipase of certain species, such as Chromobacterium viscosum lipase, Pseudomonas fluorescens lipase, Burcholderia cepacia lipase, and Rhizopus oryzae lipase, does not necessarily mean that the lipase was prepared directly from the native host species. For example, the same lipase may be produced recombinantly in another host cell.

### Immobilized Lipase

Processes for immobilizing enzymes and other proteins to insoluble supports are well-known and described in the literature. Immobilization of lipase may improve the stability of the enzyme, render it reusable, and allow products to be readily separated from the enzyme without contamination by lipase. In some embodiments, the lipase is bound to a solid support via covalent or non-covalent binding. Suitable methods of immobilization of lipase include, for example, adsorption, ionic binding, non-covalent binding, cross-linking, encapsulation, and entrapment onto hydrophobic or hydrophilic polymeric and inorganic matrices. See Ren et al., BMC Biotechnol. 11:63 (2011); Murty et al., Biotechnol. Bioprocess Eng. 7:57-66 (2002). Lipase may be immobilized by binding directly to a support material or through a linker. See, e.g., Stark and Holmberg, Biotechnol. and Bioeng. 34 (7): 942-950 (1989). Suitable methods of immobilization of enzymes onto a solid support may also include drying the enzymes onto such solid supports, e.g., beads. Any of the processes for immobilizing enzymes as discussed herein, and any additional steps, e.g., drying, may produce a dry support. The dryness of the support, e.g., bead, however, may decrease the amount of fats that are hydrolyzed as a nutritional composition flows through a hydrolysis device 20 as utilized in current systems and methods.

In some embodiments, the immobilized lipase is a microbial lipase. In some embodiments, the immobilized lipase is selected from bacterial lipases. In some embodiments, the immobilized lipase may be one or more lipases selected from Chromobacterium viscosum, Pseudomonas fluorescens, Burcholderia cepacia, and Rhizopus oryzae.

In certain embodiments, the nutritional composition may be exposed to the lipase for approximately 1, 2, 3, 4, 5, 10, 20, 30 minutes or more, as will be described in more detail below. In certain embodiments, the percent hydrolysis of triglycerides and esters in the nutritional composition may be approximately 10%, 20%, 30%, 40%, 50%, 60%, 70%, 80%, 90%, or 100% using the double-pass method described herein.

### Hydrolysis Devices Comprising Immobilized Lipase

According to various embodiments, the present disclosure provides systems and methods for preparing hydrolyzed nutritional compositions. The systems and methods can be used to expose nutritional compositions to lipases prior to consumption. The lipases may accordingly break down fats into free fatty acids and monoglycerides. The systems and methods may allow convenient means for preparing a nutritional composition. In some embodiments, the systems and methods may allow subjects, such as infants or others who consume the hydrolyzed nutritional compositions, to avoid consuming significant amounts of exogenous lipase. In some embodiments, the systems and methods may allow for production of formulas that contain monoglycerides and/or free fatty acids but do not contain any significant amount of lipase (as determined by a BCA protein quantification assay).

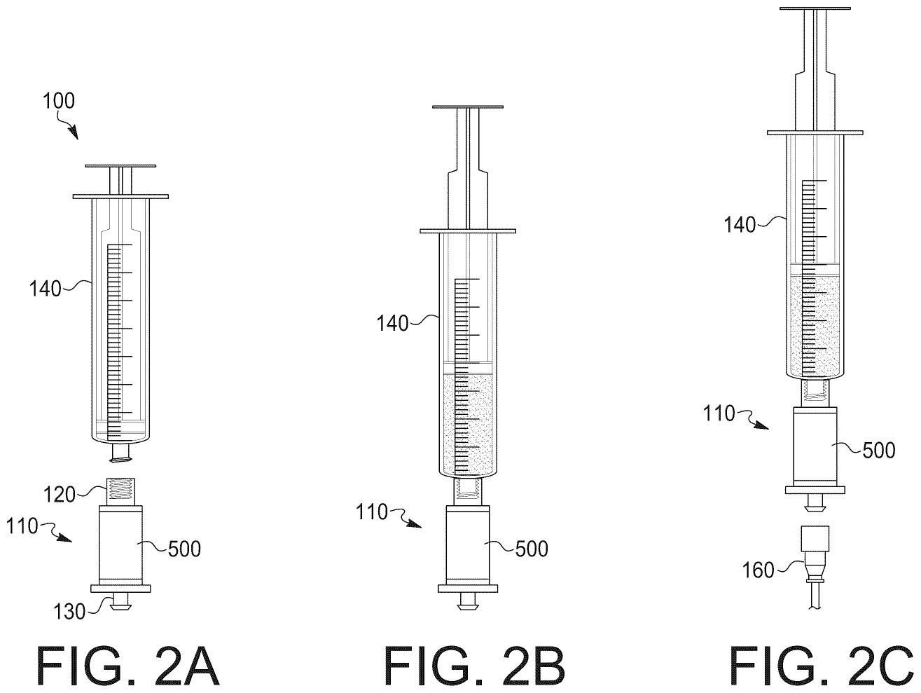

FIGS. 2A-2C, 3A-3C, and 4A-4C illustrate systems and devices according to various embodiments of the present disclosure. Hydrolysis device 110 may contain immobilized lipase, as described above. For example, lipases 500 may be immobilized on the interior wall of hydrolysis device 110, on structures such as beads contained within hydrolysis device 110, or otherwise within hydrolysis device 110. In the RELIZORB® device, lipase is immobilized to beads within the device. Further, as will be described in detail below, a nutritional composition may be passed through hydrolysis device 110 multiple times so as to facilitate hydrolysis of the nutritional composition by lipase 500 by exposing lipase 500 to the nutritional composition repeatedly. Such exposure includes pre-wetting any structures such as beads contained within hydrolysis device 110. According to methods and systems of the present disclosure, a nutritional composition may be drawn into hydrolysis device 110 using a syringe 140 to expose the nutritional composition to the lipase 500 a first time, and then may be expelled from syringe 140 and passed through hydrolysis device 110 a second time, prior to being delivered to a subject.

As shown in FIGS. 2A-2C, hydrolysis device 110 of the present disclosure may comprise a first opening 120 and a second opening 130. First opening 120 and second opening 130 may be used for ingress and egress of formula. First opening 120 and/or second opening 130 may comprise a structure configured to engage other devices that may be used for feeding or transfer of fluids. For example, first opening 120 and/or second opening 130 may include a connector such as a luer-lock connection, threads, and/or a conduit or tube that may engage a feeding tube, an enteral straw, or a syringe.

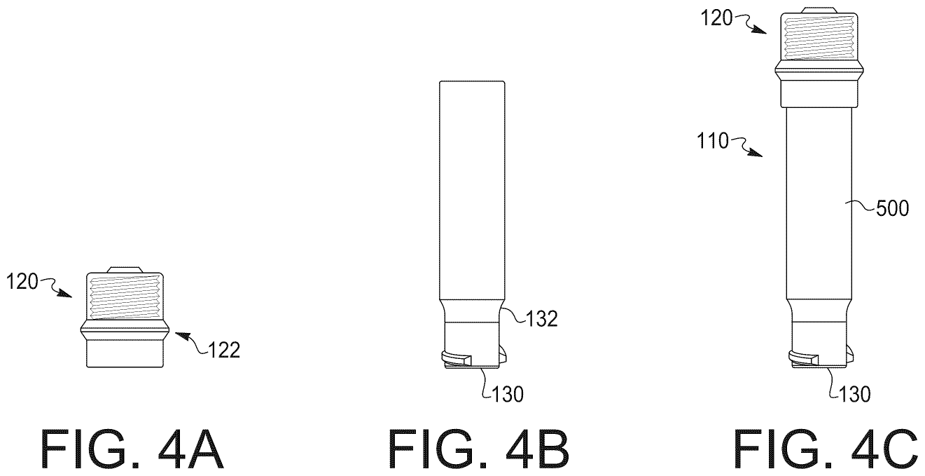

In some embodiments, e.g., referring to FIGS. 2A-2C, hydrolysis device 110 may be manufactured or formed as a single part. In other embodiments, referring to FIGS. 4A and 4B, first opening 120 and second opening 130 may be formed as separate and distinct components, wherein first opening 120 and second opening 130 may be fluidly connected to form hydrolysis device 110. First opening 120 and second opening 130 may have any appropriate configuration and/or size such that first opening 120 and second opening 130 may be fluidly coupled to form hydrolysis device 110.

In some embodiments, the volume of hydrolysis device 110 may range from about 0.5 mL to about 2 mL, from about 2 mL to about 5 mL, from about 4 mL to about 6 mL, from about 5 mL to about 8 mL, from about 5 mL to about 10 mL, from about 10 mL to about 15 mL, from about 15 mL to about 20 mL, from about 25 mL to about 30 mL, from about 0.5 mL to about 4 mL, from about 0.5 mL to about 5 mL, from about 0.5 mL to about 6 mL, from about 0.5 mL to about 8 mL, from about 0.5 mL to about 10 mL, from about 0.5 mL to about 15 mL, from about 0.5 mL to about 20 mL, from about 0.5 mL to about 25 mL, or from about 0.5 mL to about 30 mL.

Referring to FIGS. 4A and 4B, first opening 120 and/or second opening 130 may include a filter to prevent immobilized lipases 500 from exiting hydrolysis device 110, e.g., as the formula flows into hydrolysis device 110 via first opening 120 and out of hydrolysis device 110 via second opening 130. In some embodiments, first opening 120 may include filter 122 (FIG. 4A). In other embodiments, second opening 130 may include filter 132 (FIG. 4B). Filter 122, 132 may have any appropriate pore size to prevent lipases 500 and any structures lipases 500 may be immobilized to from exiting hydrolysis device 110. A pore size of filter 122, 132 may range from about 0.1 mm to about 4 mm. For example, a pore size of filter 122, 132 may range from about 0.1 mm to about 3.5 mm, about 0.1 mm to about 3 mm, about 0.1 mm to about 2.5 mm, about 0.1 mm to about 2 mm, about 0.1 mm to about 1.5 mm, about 0.1 mm to about 1 mm, about 0.5 mm to about 4 mm, about 0.5 mm to about 3.5 mm, about 0.5 mm to about 3 mm, about 0.5 mm to about 2.5 mm, about 0.5 mm to about 2 mm, about 1 mm to about 4 mm, about 1 mm to about 3.5 mm, about 1 mm to about 3 mm, about 1 mm to about 2.5 mm, about 1 mm to about 2 mm, about 1.5 mm to about 4 mm, about 1.5 mm to about 3.5 mm, about 1.5 mm to about 3 mm, about 1.5 mm to about 2.5 mm, or about 1.5 mm to about 2 mm.

Referring back to FIGS. 2A-2C, system 100 may include hydrolysis device 110 and syringe 140. Syringe 140 may be configured to attach to a first or a second opening of hydrolysis device 110. For example, an inlet portion of syringe 140 may be connected to first opening 120 of hydrolysis device 110. In some embodiments, first opening 120 may include a connector configured to connect to a portion of syringe 140. For example, first opening 120 may generally be in the shape of a cylinder, a funnel, or a truncated cone for example, and may be designed to match any suitable standardized connector, such as an ENFit™ connector. Once first opening 120 is connected to a portion of syringe 140, a fluid connection between first opening 120 and syringe 140 is formed. Syringe 140 may be used to draw a nutritional composition through hydrolysis device 110 and into syringe 140. For example, second opening 130 of hydrolysis device 110 may be used to draw a nutritional composition from a source, e.g., a container, such as a bottle, jar, vial, or bag, configured for holding a nutritional composition.

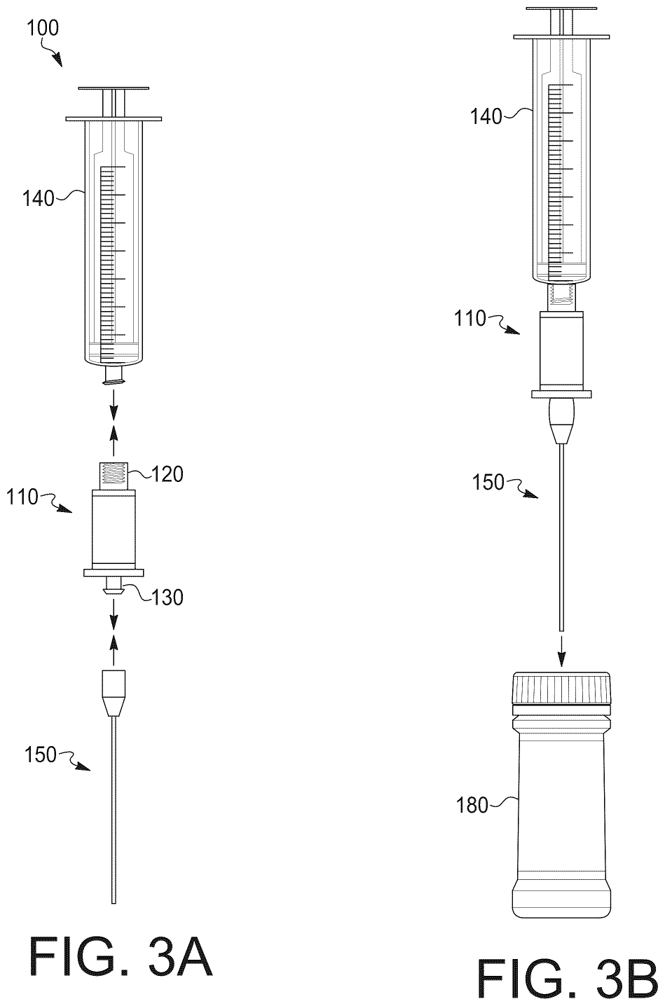

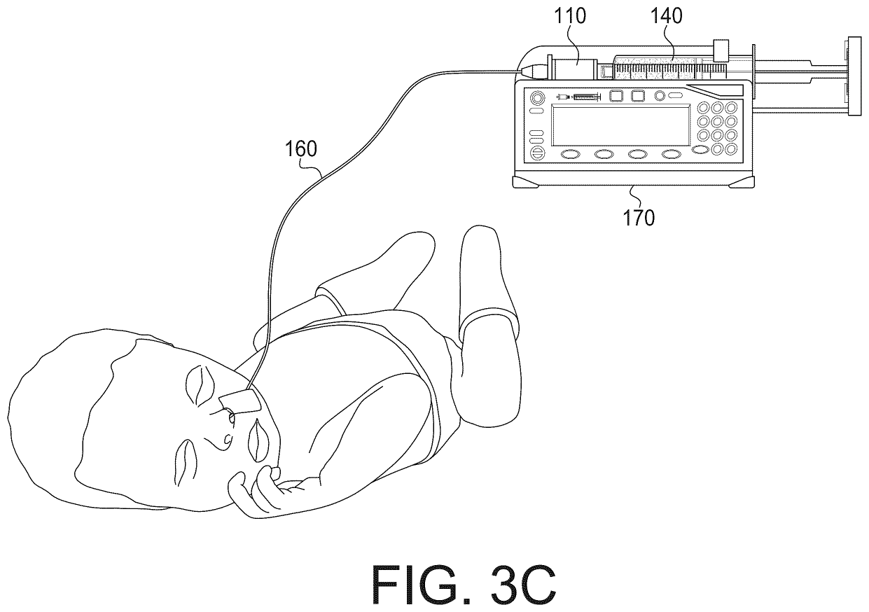

In other embodiments, system 100 may include a straw 150 (FIG. 3A). Straw 150 may be an enteral straw or any appropriate straw that may connect to second opening 130 of hydrolysis device 110. Second opening 130 may include a connector configured to connect to a portion of straw 150. For example, a portion of second opening 130 may generally be in the shape of a cylinder, a funnel, or a truncated cone, and may be designed to match any suitable standardized connector, such as an ENFit™ connector. Once second opening 130 is connected to straw 150, a fluid connection between second opening 130 and straw 150 is formed. Straw 150 may facilitate filing of syringe 140 with a nutritional composition from a source 180, which will be discussed in further detail below.

In other embodiments, system 100 may include an extension set, which may include a feeding tube 160 for feeding the nutritional composition to a subject (FIGS. 2C and 3C). Feeding tube 160 may be an enteral feeding tube, for example, a gastric, a nasogastric, a nasoduodenal, a nasojejunal, a gastrostomy, a gastrojejunostomy, a jejunostomy, a PEG tube, or a transjejunal feeding tube to feed the nutritional composition to the GI tract of a subject through, for example, the nose, mouth, stomach, or abdomen. System 100 may be used in line with current standard enteral feeding practice. In some aspects, after nutritional composition is drawn into the syringe 140 and through hydrolysis device 110 for a first time, e.g., through an enteral straw or directly through hydrolysis device 110, then feeding tube 160 may be connected to second opening 130 of hydrolysis device 110 so that when the nutritional composition is expelled from syringe 140 and back through hydrolysis device 110 for a second time, the nutritional composition may be expelled from hydrolysis device 110 into feeding tube 160. If straw 150 is used to draw the nutritional composition from the source and into hydrolysis device 110 and into syringe 140, then straw 150 may be removed after the nutritional composition fills syringe 140, and feeding tube 160 may be attached in its place.

Referring to FIG. 3C, in some aspects, system 100 may include a syringe pump 170, although use of a syringe pump 170 is optional. The flow of the nutritional composition to a subject may be controlled by syringe pump 170 of system 100. A flow rate of the nutritional composition out of syringe 140 and/or hydrolysis device 110 may be set and/or adjusted by syringe pump 170. In some embodiments, syringe pump 170 may include a processor, a display, and/or actuators (e.g. buttons, knobs, touch screen, etc.) to adjust and control the flow rate of the nutritional composition in system 100 and through hydrolysis device 110. Syringe pump 170 may be adjusted and set by a healthcare provider and/or the subject receiving the nutritional composition. Syringe pump 170 may perform continuous feeding, pulsatile feeding, intermittent feeding, bolus feeding, and/or flushing, and delivery of fluids may be set or adjusted automatically, semi-automatically, or manually.

In some embodiments, syringe pump 170 may be a smart pump. Syringe pump 170 may make automatic adjustments to the flow rate based on timing or feedback from system 100. Syringe pump 170 may include user alerts to warn when the user sets parameters for syringe pump 170 that fall outside of specified limits. Syringe pump 170 may send an alert when an actual flow rate of the nutritional composition falls outside of set parameters for syringe pump 170. The parameters may be stored in a memory of syringe pump 170, or may be entered and/or adjusted for a specific delivery regime.

### Methods of Preparing a Nutritional Composition Using a Double-Pass Method

According to various embodiments, the present disclosure also provides methods of preparing nutritional compositions, e.g., using system 100 described above.

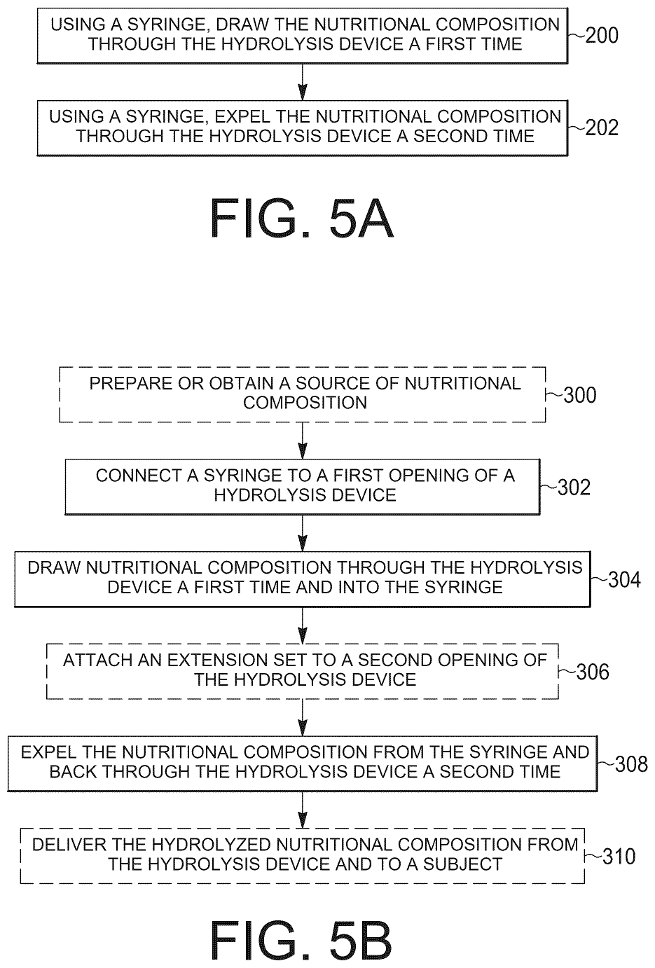

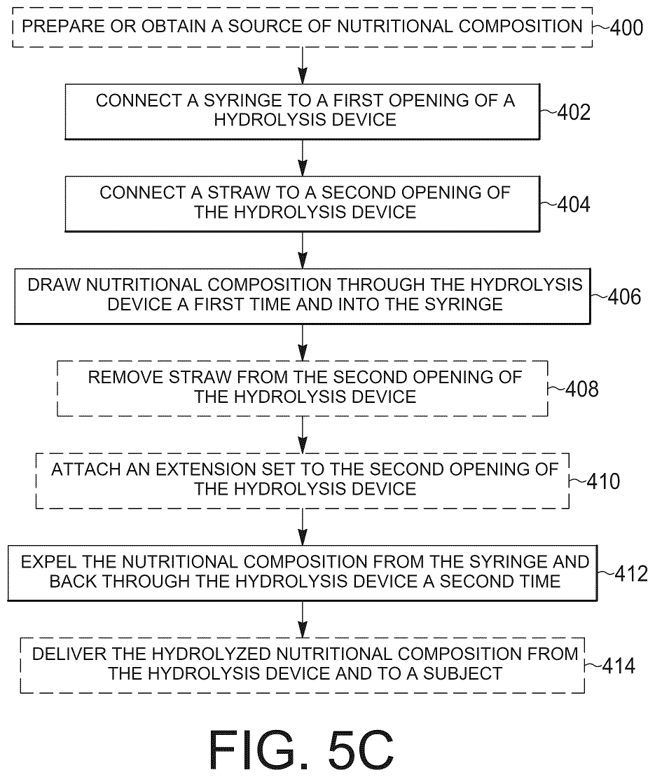

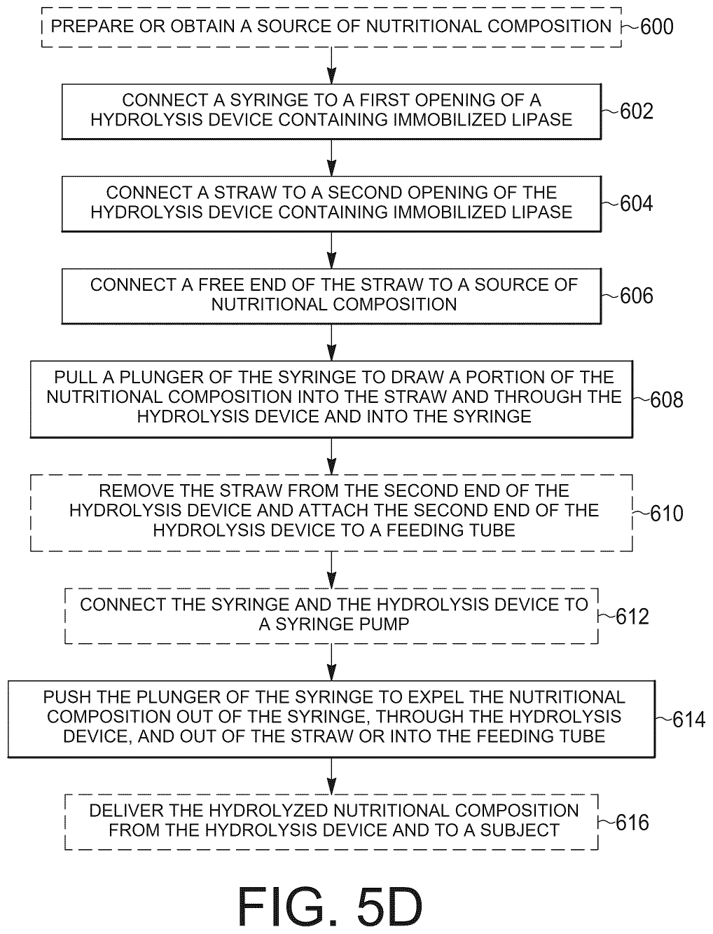

In some embodiments, a method of preparing a nutritional composition comprises exposing the nutritional composition to a lipase a first time, and then exposing the nutritional composition to the lipase a second time in order to sufficiently hydrolyze the nutritional composition, referred to herein as a double-pass method. For example, as is shown in FIG. 5A, the method may comprise step 200 of using a syringe to draw the nutritional composition through the hydrolysis device a first time to expose the nutritional composition to the lipase within the hydrolysis device a first time. The method may further include a step 202 of using the syringe to expel the nutritional composition through the hydrolysis device a second time to expose the nutritional composition to the lipase within the hydrolysis device a second time.

In some embodiments, a method of preparing a nutritional composition using hydrolysis 110 may include the steps shown in FIG. 5B. Step 300 is an optional step that may include obtaining or preparing a source of a nutritional composition. For example, step 300, if performed, may include obtaining and/or preparing a nutritional composition of a predetermined volume in a container, e.g., a bag, vial, or bottle. Step 302 may include fluidly connecting syringe 140 to a portion of hydrolysis device 110, e.g., first opening 120. This first step is depicted in FIG. 2A. Step 304 may include drawing at least a portion of the nutritional composition from the container via second opening 130 of hydrolysis device 110, such that a portion of the nutritional composition flows through hydrolysis device 110 and into the syringe barrel of syringe 140. Drawing the nutritional composition into hydrolysis device 110 and into syringe 140 may include pulling back a plunger of syringe 140, so as to draw the nutritional composition into the syringe barrel. As the nutritional composition is drawn through hydrolysis device 110 and into syringe 140, the nutritional composition passes through lipase 500 such that the nutritional composition is exposed to lipase 500 for the first time. This step is illustrated in FIG. 2B. As the nutritional composition is passed through lipase 500, which may be immobilized on a plurality of beads, the nutritional composition may wet the beads, and the fats in the nutritional composition may contact the immobilized lipase. Optional step 306 may include attaching a portion of hydrolysis device 110 to a patient extension set, e.g., feeding tube 160 or other feeding mechanism. This step is illustrated in FIG. 2C. Step 308 may include expelling the nutritional composition from syringe 140 and into the hydrolysis device 110 a second time, e.g., by pushing a plunger of syringe 140 into the syringe barrel and towards hydrolysis device 110, such that the nutritional composition is exposed to lipase 500 for a second time. During step 308, the hydrolyzed nutritional composition may also be expelled from hydrolysis device 110 via second opening 130 and into the extension set, e.g., feeding tube 160, if an extension set is attached at step 306. In other embodiments, hydrolyzed nutritional composition may be expelled from the hydrolysis device 110 into a container or other feeding mechanism for administration to a subject immediately or after a given amount of time. Optional step 310 may include delivering the hydrolyzed nutritional composition to a subject, e.g., via feeding tube 160 or other feeding mechanism. In methods where hydrolysis device 110 is connected to a feeding mechanism, e.g., syringe pump 170, as shown in FIG. 4B, then one or both of steps 308 and 310 may be performed by the syringe pump 170. In some aspects, steps 308 and 310 may be performed substantially simultaneously.

In another embodiment, a method of preparing a nutritional composition using hydrolysis device 110 may include the steps shown in FIG. 5C. Step 400 is an optional step of preparing or obtaining a source of a nutritional composition. For example, step 400, if performed, may include obtaining and/or preparing a nutritional composition of a predetermined volume in a container, e.g., a bag, vial, or bottle. Step 402 may include fluidly connecting syringe 140 to a portion of hydrolysis chamber 110, e.g., first opening 120. Step 404 may include fluidly connecting straw 150 to a portion of hydrolysis device 110, e.g., second opening 130. Steps 402 and 404 are depicted in FIG. 3A, described above. Step 406 may include drawing at least a portion of the nutritional composition from the container via straw 150, such that a portion of the nutritional composition flows through straw 150, through hydrolysis device 110, and into the syringe barrel of syringe 140. As the nutritional composition is drawn through hydrolysis device 110 and into syringe 140, the nutritional composition passes through lipase 500 such that the nutritional composition is exposed to lipase 500 for the first time. Optional step 408 may include removing straw 150. Optional step 410 may include attaching a portion of hydrolysis device 110 to a patient extension set, e.g., feeding tube 160 or other feeding mechanism. Step 412 may include expelling the nutritional composition from syringe 140, e.g., by pushing a plunger of syringe 140 into the syringe barrel and towards hydrolysis device 110, such that the nutritional composition is exposed to lipase 500 for a second time. During step 412, the hydrolyzed nutritional composition may also exit hydrolysis device 110 via second opening 130, e.g., through either straw 150 or the extension set, e.g., feeding tube 160. Optional step 414 may include delivering the hydrolyzed nutritional composition to a subject via feeding tube 160 or other feeding mechanism. In methods where hydrolysis device 110 is connected to a feeding mechanism, e.g., a syringe pump 170, as shown in FIG. 3C, then one or both of steps 412 and 414 may be performed by syringe pump 170. In some aspects, steps 412 and 414 may be performed substantially simultaneously.

In another embodiment, a method of preparing a nutritional composition using hydrolysis device 110 may include the steps shown in FIG. 5D. Step 600 is an optional step of preparing or obtaining a source of a nutritional composition. For example, step 600, if performed, may include obtaining and/or preparing a nutritional composition of a predetermined volume in a container, e.g., a bag, vial, or bottle. Step 602 may include fluidly connecting syringe 140 to a portion of hydrolysis chamber 110, e.g., first opening 120, wherein hydrolysis chamber 110 contains immobilized lipase. Step 604 may include fluidly connecting straw 150 to a portion of hydrolysis device 110, e.g., second opening 130. Steps 602 and 604 are depicted in FIGS. 2A and 3A, described above. Step 606 may include contacting a free end of straw 150 to a source of nutritional composition. Step 606 is depicted in FIG. 3B, described above. Step 608 may include pulling a plunger of syringe 140 out of the syringe barrel to draw a portion of the nutritional composition from the container via straw 150, such that a portion of the nutritional composition flows through straw 150, through hydrolysis device 110, and into the syringe barrel of syringe 140. As the nutritional composition is drawn through hydrolysis device 110 and into syringe 140, the nutritional composition passes through lipase 500 such that the nutritional composition is exposed to lipase 500 for the first time. Optional step 610 may include removing straw 150 from hydrolysis device 110, e.g., from second opening 130, and attaching second opening 130 of hydrolysis device to feeding tube 160. Optional step 612 may include connecting syringe 140 and hydrolysis device 110 to syringe pump 170. Step 612 may further include setting a rate of delivery for syringe pump 170. Step 614 may include pushing the plunger of syringe 140 to expel the nutritional composition out of syringe 140, such that a portion of the nutritional composition flows through the syringe barrel of syringe 140, through hydrolysis device 110, and out of straw 150 or into feeding tube 160. Pushing of the plunger may be controlled by syringe pump 170, which may push the plunger to expel the nutritional composition at a rate that a user may set for syringe pump 170. Optional step 616 may include delivering the hydrolyzed nutritional composition to a subject via feeding tube 160 or other feeding mechanism. In methods where hydrolysis device 110 is connected to a feeding mechanism, e.g., syringe pump 170, as shown in FIG. 3C, then one or both of steps 614 and 616 may be performed by a rate controlled by syringe pump 170. In some aspects, steps 614 and 616 may be performed substantially simultaneously.

In methods where hydrolysis device 110 is connected to a feeding mechanism, e.g., syringe pump 170, additional steps may include programing syringe pump 170 and/or initiating feeding. In such embodiments, syringe pump 170 may be programed to dispense the nutritional composition from syringe 140, e.g., by pushing a plunger of syringe 140 into the syringe barrel and towards hydrolysis device 110.

In the exemplary double pass methods described above, the nutritional composition passes through hydrolysis device 110 and lipase 500 more than once to increase the exposure of the nutritional composition to the lipase. For example, a first pass is made when the nutritional composition is drawn through hydrolysis device 110 and into syringe 140, and a second pass is made when the nutritional composition is expelled from syringe 140, through hydrolysis device 110, and potentially to a patient extension set, e.g., feeding tube 160 or other feeding mechanism. Multiple-pass methods may be used for patients generally or may be used in scenarios in which there is a limitation on the total number of beads with immobilized lipase that may be used each day by a patient, or a limitation on the total number of beads with immobilized lipase that may be used at the same time. For example, regulatory restrictions may limit the total amount of beads with immobilized lipase to which a subject may be exposed in a single day. The reduction in beads used per feeding may, however, lower hydrolysis efficiency of hydrolysis device 110. As mentioned above, different compositions of commercially available nutritional formulas may also impact the extent of hydrolysis during preparation and feeding. For example, compositions of nutritional formulas may be such that components such as fats and nutrients may adhere to portions of system 100, including, e.g., hydrolysis device 110, syringe 140, and/or straw 150, such that the amount of nutritional formula that may be hydrolyzed in reduced, further reducing hydrolysis efficiency in hydrolysis device 110. Similar issues may occur with milk. This reduction in hydrolysis efficiency may be offset, or at least partially offset, by using a multi-pass method to boost percent hydrolysis by increasing residence time and/or exposure to the beads.

As discussed above, the beads utilized in the disclosed systems and methods may be extremely dry. The dryness of the beads may decrease the amount of fats hydrolyzed as the nutritional composition flows through hydrolysis devices utilized in current systems and methods. In the exemplary double pass methods described above, drawing the nutritional composition through the hydrolysis device a first time may serve to pre-wet any structures, such as beads, that the lipase may be immobilized to. This pre-wetting of the beads may increase the total amount of fats that are hydrolyzed after the nutritional composition is drawn through the hydrolysis device the second time.

Further, use of system 100 may itself address the issue of fat loss to the system compared to the system of FIG. 1, because system 100 includes much less tubing for the fat and nutrients to adhere to. Thus, multiple-pass methods may be useful during the preparation and delivery of a nutritional composition, for example, to increase percent hydrolysis without introducing significant additional steps or changes to the method of using hydrolysis device 110, particularly for infant nutritional formula preparation.

In some aspects, hydrolysis device 110 may be reusable, such that a single hydrolysis device 110 may be used multiple times. In other embodiments, hydrolysis device 110 may be disposable, such that after hydrolysis device 110 is used, it may be disposed. In circumstances where subjects may need multiple feedings in a designated period of time, multiple hydrolysis device may be used and any of the methods described above may be repeated until the subject is properly treated. In some embodiments, at least one hydrolysis device 110 may be utilized in a 24 hour period, at least two hydrolysis devices 110 may be utilized in a 24 hour period, at least three hydrolysis devices 110 may be utilized in a 24 hour period, at least four hydrolysis devices 110 may be utilized in a 24 hour period, at least five hydrolysis devices 110 may be utilized in a 24 hour period, at least six hydrolysis devices 110 may be utilized in a 24 hour period, at least seven hydrolysis device 110 may be utilized in a 24 hour period, or at least eight hydrolysis devices 110 may be utilized in a 24 hour period. For example, up to eight hydrolysis devices 110 may be used per day to support more frequent feedings common in neonates and infants.

As described above, a nutritional composition may be directed through hydrolysis device 110 via use of syringe 140 or by syringe pump 170. In some embodiments, a nutritional composition may be directed through hydrolysis device 110 at a flow rate ranging from about 0.01 mL/hour to about 1.5 mL/hour. For example, the flow rate may range from about 0.01 mL/hour to about 1.25 mL/hour, from about 0.01 mL/hour to about 1 mL/hour, from about 0.05 mL/hour to about 1.5 mL/hour, from about 0.05 mL/hour to about 1.25 mL/hour, from about 0.05 mL/hour to about 1 mL/hour, from about 0.1 mL/hour to about 1.5 mL/hour, from about 0.1 mL/hour to about 1 mL/hour, from about 0.2 mL/hour to about 1.5 mL/hour, from about 0.2 mL/hour to about 1.25 mL/hour, from about 0.2 mL/hour to about 1 mL/hour, from about 0.3 mL/hour to about 1.5 mL/hour, from about 0.3 mL to about 1.25 mL/hour, from about 0.3 mL to about 1 mL/hour, from about 0.4 mL to about 1.5 mL/hour, from about 0.4 mL/hour to about 1.25 mL/hour, from about 0.5 mL/hour to about 1.5 mL/hour, from about 0.5 mL/hour to about 1.25 mL/hour, from about 0.5 mL/hour to about 1 mL/hour, from about 0.6 mL/hour to about 1.5 mL/hour, from about 0.6 mL/hour to about 1.25 mL/hour, from about 0.6 mL/hour to about 1 mL/hour, from 0.7 mL/hour to about 1.5 mL/hour, from about 0.7 mL/hour to about 1.25 mL/hour, from about 0.7 mL/hour to about 1 mL/hour, from about 0.8 mL/hour to about 1.5 mL/hour, from about 0.8 mL/hour to about 1.25 mL/hour, from about 0.8 mL/hour to about 1 mL/hour, from about 0.9 mL/hour to about 1.5 mL/hour, from about 0.9 mL/hour to about 1.25 mL/hour, from about 0.9 mL/hour to about 1 mL/hour, from about 5 mL/hour to about 20 mL/hour, from about 20 mL/hour to about 75 mL/hour, from about 50 mL/hour to about 100 mL/hour, from about 100 mL/hour to about 400 mL/hour, from about 100 mL/hour to about 450 mL/hour, from about 200 mL/hour to about 400 mL/hour, or from about 300 mL/hour to about 450 mL/hour.

In some embodiments, the time needed to deliver the total amount of nutritional composition through hydrolysis device 110, i.e., a feeding time of nutritional composition, may depend on the flow rate, the volume of hydrolysis device 110, and/or the total volume of nutritional composition to be delivered to the subject. For example, a faster flow rate and/or a larger volume of hydrolysis device 110 may allow a predetermined volume of nutritional composition to flow through system 100 and/or hydrolysis device 110 for a shorter feeding time.

In some embodiments, the feeding time may depend on the need or enteral feeding practice suitable to the subject. In some embodiments, the feeding time may range, for example, from about a few seconds to a few minutes, from about a few minutes to about 30 minutes, from about 30 minutes to about an hour, from about an hour to about 4 hours, from about 4 hours to about 10 hours, from about 10 hours to about 12 hours, from about 12 hours to about 18 hours, from about 18 hours to about 24 hours, from about 12 hours to about 24 hours, or continuous. In some embodiments, a shorter feeding time may be preferable for subjects in need of nutritional composition.

In some embodiments, the duration of the first pass of the nutritional composition through hydrolysis device 110 and into syringe 140 may be up to about 5 minutes. Accordingly, the nutritional composition may be exposed to immobilized lipase within device 110 as it passes through device 110 over the course of 5 minutes. For example, the duration of the first pass may be at least about 1 second, at least about 2 seconds, at least about 3 seconds, at least about 5 seconds, at least about 10 seconds, at least about 15 seconds, at least about 30 seconds, at least about 1 minute, at least about 2 minutes, at least about 3 minutes, or at least about 5 minutes. In some embodiments, the first pass of the nutritional composition through hydrolysis device 110 and into syringe 140 may range from about 1 second to about 5 minutes, about 1 second to about 3 minutes, about 1 second to about 2 minutes, about 1 second to about 1 minute, about 1 second to about 30 seconds, about 1 second to about 15 seconds, about 1 second to about 10 seconds, about 1 second to about 5 seconds, about 2 seconds to about 5 minutes, about 2 seconds to about 3 minutes, about 2 seconds to about 2 minutes, about 2 seconds to about 1 minute, about 2 seconds to about 30 seconds, about 2 seconds to about 15 seconds, about 2 seconds to about 10 seconds, about 2 seconds to about 5 seconds, about 3 seconds to about 5 minutes, about 3 seconds to about 3 minutes, about 3 seconds to about 2 minutes, about 3 seconds to about 1 minute, about 3 seconds to about 30 seconds, about 3 seconds to about 15 seconds, about 3 seconds to about 10 seconds, about 3 seconds to about 5 seconds, about 5 seconds to about 5 minutes, about 5 seconds to about 3 minutes, about 5 seconds to about 2 minutes, about 5 seconds to about 1 minute, about 5 seconds to about 30 seconds, about 5 seconds to about 15 seconds, about 5 seconds to about 10 seconds, about 10 seconds to about 5 minutes, about 10 seconds to about 3 minutes, about 10 seconds to about 2 minutes, about 10 seconds to about 1 minute, about 10 seconds to about 30 seconds, about 10 seconds to about 15 seconds, about 15 seconds to about 5 minutes, about 15 seconds to about 3 minutes, about 15 seconds to about 2 minutes, about 15 seconds to about 1 minute, about 15 seconds to about 30 seconds, about 30 seconds to about 5 minutes, about 30 seconds to about 3 minutes, about 30 seconds to about 2 minutes, or about 30 seconds to about 1 minute. The duration of the first pass may be equivalent to the amount of time the nutritional composition is exposed to lipase during the first pass.

In some embodiments, the duration of the second pass of the nutritional composition from syringe 140 back through hydrolysis device 110 a second time may be at least about 1 second, at least about 2 seconds, at least about 3 seconds, at least about 5 seconds, at least about 10 seconds, at least about 15 seconds, at least about 30 seconds, at least about 1 minute, at least about 2 minutes, at least about 3 minutes, or at least about 5 minutes. In some embodiments, the second pass of the nutritional composition through hydrolysis device 110 and into syringe 140 may range from about 1 second to about 5 minutes, about 1 second to about 3 minutes, about 1 second to about 2 minutes, about 1 second to about 1 minute, about 1 second to about 30 seconds, about 1 second to about 15 seconds, about 1 second to about 10 seconds, about 1 second to about 5 seconds, about 2 seconds to about 5 minutes, about 2 seconds to about 3 minutes, about 2 seconds to about 2 minutes, about 2 seconds to about 1 minute, about 2 seconds to about 30 seconds, about 2 seconds to about 15 seconds, about 2 seconds to about 10 seconds, about 2 seconds to about 5 seconds, about 3 seconds to about 5 minutes, about 3 seconds to about 3 minutes, about 3 seconds to about 2 minutes, about 3 seconds to about 1 minute, about 3 seconds to about 30 seconds, about 3 seconds to about 15 seconds, about 3 seconds to about 10 seconds, about 3 seconds to about 5 seconds, about 5 seconds to about 5 minutes, about 5 seconds to about 3 minutes, about 5 seconds to about 2 minutes, about 5 seconds to about 1 minute, about 5 seconds to about 30 seconds, about 5 seconds to about 15 seconds, about 5 seconds to about 10 seconds, about 10 seconds to about 5 minutes, about 10 seconds to about 3 minutes, about 10 seconds to about 2 minutes, about 10 seconds to about 1 minute, about 10 seconds to about 30 seconds, about 10 seconds to about 15 seconds, about 15 seconds to about 5 minutes, about 15 seconds to about 3 minutes, about 15 seconds to about 2 minutes, about 15 seconds to about 1 minute, about 15 seconds to about 30 seconds, about 30 seconds to about 5 minutes, about 30 seconds to about 3 minutes, about 30 seconds to about 2 minutes, or about 30 seconds to about 1 minute. In some aspects, the duration of the second pass may be equivalent to the duration of the feeding to a patient. In such instances, the duration of the second pass may be up to about 15 minutes, up to about 30 minutes, up to about 45 minutes, up to about 60 minutes, up to about 2 hours, up to about 4 hours, up to about 6 hours, up to about 12 hours, or up to about 24 hours. The duration of the second pass may be equivalent to the amount of time the nutritional composition is exposed to immobilized lipase within device 110 during the second pass.

In some embodiments, a given portion of the liquid nutritional composition may be exposed to lipase for at least about 2 seconds, at least about 3 seconds, at least about 5 seconds, at least about 10 seconds, at least about 15 seconds, at least about 30 seconds, at least about 1 minute, at least about 2 minutes, at least about 3 minutes, at least about 5 minutes, at least about 8 minutes, at least about 10 minutes, at least about 15 minutes, at least about 30 minutes, at least about 45 minutes, or at least about 60 minutes prior to ingestion. In some embodiments, the liquid nutritional composition is exposed to lipase for a total duration of no more than about 2 seconds, no more than about 3 seconds, no more than about 5 seconds, about 10 seconds, no more than about 15 seconds, no more than about 30 seconds, no more than about 1 minute, no more than about 2 minutes, no more than about 3 minutes, no more than about 5 minutes, no more than about 8 minutes, no more than about 10 minutes, no more than about 15 minutes, no more than about 30 minutes, no more than about 45 minutes, no more than about 60 minutes. In some embodiments, use of the double-pass method results in a nutritional composition in which at least about 10%, at least about 15%, at least about 20%, at least about 30%, at least about 40%, at least about 50%, at least about 60%, at least about 70%, at least about 80%, at least about 85%, at least about 90%, at least about 95%, or about 100% of the total fatty acids in the nutritional composition is in the form of monoglycerides and/or free fatty acids.

In some embodiments, a method of preparing a nutritional composition comprises exposing a liquid nutritional composition to a device as described herein.

Patients suffering from EPI (insufficient production of exocrine pancreatic enzymes) and/or gastrointestinal or liver dysfunction have a reduced ability to hydrolyze and/or absorb triglycerides. As a result, they might have maldigestion and malabsorption of lipids, which may lead to reduced caloric intake, significant weight loss, fatty acids deficiencies, and/or GI symptoms, and may be deprived of the benefits associated with ingestion of fatty acids. System 100 and hydrolysis device 110 may be used for feeding a nutritional composition of the present disclosure having pre-hydrolyzed triglycerides to patients having compromised pancreatic output. For example, system 100 and hydrolysis device 110 may be used to increase the intake of fatty acids in the plasma of these patients. In some embodiments, since healthy subjects may also benefit from increased absorption of fatty acids, e.g., by reducing the risk of cardiovascular disease, system 100 and hydrolysis device 110 may be used for feeding a healthy subject a nutritional composition according to the present disclosure. In some embodiments, system 100 and hydrolysis device 110 may be used to increase the intake of fatty acids in the plasma of infants, aging adults, or people with acute or chronic conditions that may impact fat hydrolysis and/or absorption.

The systems, devices, methods, and nutritional compositions discussed herein may be provided to a subject having at least one of Alzheimer's disease, bipolar disorder, depression, sepsis, acute respiratory stress, cancer, cardiovascular disease, stroke, Parkinson's disease, schizophrenia, diabetes, multiple sclerosis, malnutrition, impaired GI function, rheumatoid arthritis, systemic lupus erythematosus, inflammatory bowel disease, short bowel syndrome, irritable bowel disorder, Systemic Inflammatory Response Syndrome, celiac disease, Crohn's disease, Cystic Fibrosis, Zollinger-Ellison syndrome, hypertriglyceridemia, neoplasms, hemochromatosis, primary sclerosing cholangitis, primary biliary cirrhosis, diarrhea, Shwachman's syndrome, trypsinogen deficiency, enterokinase deficiency, chylothorax, isolated deficiency of lipase, premature birth, necrotizing enterocolitis, pancreatic insufficiency, pancreatitis, malabsorption, compromised pancreatic output, a reduced ability to hydrolyze triglycerides or esters, or a reduced ability to absorb triglycerides or esters. The systems, devices, methods, and nutritional compositions discussed herein may be provided to a subject who is an infant, including a pre-term infant, an infant in the NICU, an infant who is failure to thrive, or an infant who is otherwise experiencing difficulty with the digestion of food, etc.

As described above, delivering a nutritional composition using system 100 with hydrolysis device 110 may allow for effective hydrolysis of a nutritional composition prior to ingestion and the delivery of hydrolyzed and absorbable fatty acids to the GI tract of a subject. Also, use of hydrolysis device 110 with system 100 and the methods described herein may be compatible with a wide range of complex, commercially available nutritional formulas and may not negatively affect other nutrients in the nutritional formula or milk.

### EXAMPLE 1

Experiments were performed to compare the hydrolysis activity of lipase on fats in a nutritional formula using a method and a double-pass method, as disclosed herein. The experiments were performed using Extensive HA® infant formula, available from Nestle, which is a formula that is considered harder to hydrolyze using the traditional single-pass method.

During the single-pass method, 500 mL of the infant formula was pushed through a hydrolysis device at a rate of 400 mL/hour (about 7 mL/min), via an enteral feeding pump. The single-pass method was also analyzed utilizing a syringe as a source of fluid, instead of an enteral feeding pump. Sixty mL syringes were used, and the syringes were refilled 5 times in order to achieve a volume of 250 mL of the infant formula. Nutritional formula contained in a syringe was expelled from the syringe and through a hydrolysis device.

To analyze the double-pass method, a hydrolysis device was attached to a syringe, as described above. Sixty mL syringes were used, and the syringes were refilled 5 times in order to achieve a volume of 250 mL of the infant formula. For the first pass, the infant formula was drawn into the syringe for a total time of about 30 seconds, such that the infant formula passed through the hydrolysis device and was exposed to the lipase within for about 30 seconds. The infant formula was then pushed manually from the syringe and back through the hydrolysis device and lipase, at a rate of 400 mL/hour (7 mL/min).

Each of the conditions was tested three times. The results in Table 1 show the percent hydrolysis for each of the three runs. Free fatty acids in the hydrolyzed nutritional formula were measured using a colorimetric assay. Table 1 shows an average hydrolysis of about 57% for the single-pass utilizing a syringe pump and an average hydrolysis of about 59% for the single-pass utilizing a syringe, compared to an average hydrolysis of about 84% for the double-pass method utilizing a syringe. When using twice the serving size of infant formula, the single-pass method of about 500 mL of infant formula via a syringe pump had an average hydrolysis of about 57% compared to the double-pass method of 250 ml of infant formula, which had an average hydrolysis of about 84%.

TABLE 1

| | | | % Hydrolysis |
|---|---|---|---|
| Test Condition | Run | % Hydrolysis | Average (n = 3) |
| 2 servings (~500 mL), | 1 | 57 | 57 |
| 400 mL/hr pump bolus | 2 | 58 | |
| (single pass) | 3 | 58 | |
| 1 serving (~250 mL), | 1 | 60 | 59 |
| syringe push bolus - single | 2 | 57 | |
| pass | 3 | 59 | |
| 1 serving (~250 mL), | 1 | 78 | 84 |
| syringe push bolus - double | 2 | 85 | |
| pass | 3 | 90 | |

### EXAMPLE 2

Experiments were performed to compare the hydrolysis activity of immobilized lipase on fats in a nutritional formula using a single-pass method and a double-pass method, as disclosed herein. The experiments were performed using Similac® Advance OptiGRO infant formula, which is a formula that is considered harder to hydrolyze using the traditional single-pass method, and two different lots of hydrolysis devices. The hydrolysis devices included about 400 mg of immobilized lipase.

To test the single-pass method, 50 mL of the infant formula was pushed from a syringe through a hydrolysis device at a rate of 120 mL/hour.

To test the double-pass method, a hydrolysis device was attached to a syringe, as described above, and 50 mL of the infant formula was drawn into a syringe over a total duration of 60 seconds. The syringe and hydrolysis device was then installed in a syringe pump for delivery of the infant formula. The syringe pump pushed the infant formula from the syringe and back through the hydrolysis device and lipase, at a rate of 120 mL/hour.

Each of the conditions was tested three times. The results in Table 2 show the percent hydrolysis for each of the three runs. Free fatty acids in the hydrolyzed nutritional formula were measured using a colorimetric assay. Table 2 shows an increase in hydrolysis using the double-pass method with the hydrolysis devices when comparing the single-pass and double pass methods. Specifically, the single-pass method using hydrolysis device lot A produced an average hydrolysis of about 34% and 30%, while the double-pass method using hydrolysis device lot B produced an average hydrolysis of about 52%.

TABLE 2

| Hydrolysis | | | | % Hydrolysis |
|---|---|---|---|---|
| Device | | | % | Average |
| Lot | Test Condition | Run | Hydrolysis | (n = 3) |
| A | 50 mL at 120 mL/hr | 1 | 36 | 34 |
| | syringe push bolus- | 2 | 31 | |
| | single pass | 3 | 35 | |
| A | 50 mL at 120 mL/hr | 1 | 53 | 52 |
| | syringe push bolus- | 2 | 52 | |
| | double pass | 3 | 52 | |
| B | 50 mL at 120 mL/hr | 1 | 30 | 30 |
| | syringe push bolus- | 2 | 32 | |
| | single pass | 3 | 29 | |
| B | 50 mL at 120 mL/hr | 1 | 52 | 52 |
| | syringe push bolus- | 2 | 53 | |
| | double pass | 3 | 50 | |

### EXAMPLE 3

Experiments were performed to compare the hydrolysis activity of lipase on fats in a nutritional formula using a single-pass method and a double-pass method, as disclosed herein. The experiments were performed using two enteral formulas, TwoCal® HN and Pivot® 1.5 Cal, both available from Abbott Nutrition, and both considered hard to hydrolyze nutritional formulas using the single-pass method, and hydrolysis devices.

During the single-pass method, a syringe with a volume of 60 mL was filled with 50 mL of enteral formula 5 times to achieve a total volume of 250 mL. The enteral formula was expelled from the syringe and through a hydrolysis device at a rate of 400 mL/hour (7 mL/min).

To analyze the double-pass method, a hydrolysis device was attached to a syringe, and enteral formula was drawn into the syringe, such that the enteral formula passed through the hydrolysis device and was exposed to the lipase within. The enteral formula was then pushed manually from the syringe and back through the hydrolysis device and lipase, at a rate of 400 mL/hour (7 mL/min).

Each of the conditions was tested three times. The results in Table 3 show the percent hydrolysis for each of the three runs. Free fatty acids in the hydrolyzed nutritional formula were measured using a colorimetric assay. Table 3 shows an increase in hydrolysis for both enteral formulas when comparing the single-pass and double pass methods. Specifically, the single-pass method using TwoCal® HN produced an average hydrolysis of about 32%, while the double-pass method using TwoCal® HN produced an average hydrolysis of about 42%. Further, the single-pass method using Pivot® 1.5 Cal produced an average hydrolysis of about 64% while the double-pass method using Pivot® 1.5 Cal produced an average hydrolysis of about 85%.

TABLE 3

| | | | | % |
|---|---|---|---|---|
| | | | | Hydrolysis |
| | | | % | Average |
| Formula | Test Condition | Run | Hydrolysis | (n = 3) |
| TwoCal ® | 250 mL at | 1 | 34 | 32 |
| HN | ~400 mL/hr | 2 | 29 | |
| | syringe push bolus- | 3 | 32 | |
| | single pass | | | |
| TwoCal ® | 250 mL at | 1 | 40 | 42 |
| HN | ~400 mL/hr | 2 | 44 | |
| | syringe push bolus- | 3 | 41 | |
| | double pass | | | |
| Pivot ® 1.5 | 250 mL at | 1 | 63 | 64 |
| Cal | ~400 mL/hr | 2 | 58 | |
| | syringe push bolus- | 3 | 69 | |
| | single pass | | | |
| Pivot ® 1.5 | 250 mL at | 1 | 90 | 85 |
| Cal | ~400 mL/hr | 2 | 85 | |
| | syringe push bolus- | 3 | 79 | |
| | double pass | | | |

### EXAMPLE 4

Experiments were performed to compare the hydrolysis activity of lipase on fats in a nutritional formula using a single-pass method and a double-pass method, as disclosed herein. The experiments were performed using Alfamino® infant formula, available from Nestle, Boost® Kid Essentials™ 1.0, available from Nestle, Boost® Kid Essentials™ 1.5, available from Nestle, Enfamil® Enspire Optimum™, available from Mead Johnson, Extensive HA® infant formula, available from Nestle, Nutramigen®, available from Mead Johnson, Osmolite® 1.2, available from Abbott Nutrition, Osmolite® 1.5, available from Abbott Nutrition, Pivot® 1.5, available from Abbott Nutrition, PediaSure® 1.0, available from Abbott Nutrition, Similac® Advance® available from Abbott Nutrition, Similac® Alimentum®, available from Abbot Nutrition, Similac® Pure Bliss®, available from Abbott Nutrition, and Similac® 360 Total Care®, available from Abbott Nutrition. These formulas are generally considered harder to hydrolyze using the traditional single-pass method.

For the single-pass method, five syringes were prepared with 50 mL of formula in each syringe to achieve a total volume of 250 mL of the infant formula. For each run, the contents of all five syringes were pushed from the syringe through a hydrolysis device via a syringe pump at a rate of 400 mL/hour (about 7 mL/min), to simulate the delivery of 250 ml of the infant formula.

To analyze the double-pass method, a hydrolysis device was connected to a syringe, and 50 mL of formula was drawn through the hydrolysis device and into the syringe for a total duration of 30 seconds or 1 minute, such that the overall time of exposure to immobilized lipase for the entire volume of infant formula passed through the hydrolysis device was 30 seconds or 1 minute, respectively. The hydrolysis device and syringe were then connected to a syringe pump, and the infant formula was pushed from the syringe and back through the hydrolysis device at a rate of 400 mL/hour (7 mL/min). For each run, five syringes were prepared with the first pass of the infant formula through the hydrolysis device into the syringe and then expelled from the syringe and back through the hydrolysis device to simulate the delivery of 250 mL of the infant formula.

For Extensive HA®, Nutramigen®, and Similac® Advance®, the first pass of the infant formula was for a duration of 30 seconds, while for Alfamino® infant formula, Boost® Kid Essentials™ 1.0, Boost® Kid Essentials™ 1.5, Enfamil® Enspire Optimum™, Osmolite® 1.2, Osmolite® 1.5, PediaSure® 1.0, Pivot® 1.5, Similac® 360 Total Care®, Similac® Alimentum®, and Similac® Pure Bliss®, the first pass of the infant formula was for a duration of 1 minute.

Alfamino® infant formula, Enfamil® Enspire Optimum™, Similac® 360 Total Care®, Similac® Alimentum®, and Similac® Pure Bliss®, were tested twice each for the single pass and the double pass. Boost® Kid Essentials™ 1.0, Boost® Kid Essentials™ 1.5, Extensive HAR, Nutramigen®, Osmolite® 1.2, Osmolite® 1.5, PediaSure® 1.0, Pivot® 1.5, and Similac® Advance®, were tested three times each for the single pass and the double pass methods.

The results in Table 4 show the percent hydrolysis for each of the individual runs, as well as an average of the runs, for the single pass and double pass methods. Free fatty acids in the hydrolyzed nutritional formula were measured using a colorimetric assay. Eleven of the 14 formulas showed an increase in hydrolysis of greater than 10% with use of the double pass method. T-tests were conducted for 9 of the 14 formulas to determine statistical significance (P value <0.05), where the 9 formulas were tested three times for each of the single pass and double pass methods. Five out of the 9 formulas tested three times for each of the single pass and double pass methods showed a statistically significant increase in hydrolysis with use of the double pass method—Boost® Kid Essentials™ 1.5, Extensive HA®, Osmolite® 1.5, PediaSure® 1.0, and Pivot® 1.5.

TABLE 4

| | | | | | | % Difference |
|---|---|---|---|---|---|---|
| | Single Pass | | Double Pass | | P | from Single Pass |
| Formula | % Hydrolysis | | % Hydrolysis | | Value | to Double Pass |
| Alfamino ® infant | 36 | 35 | 53 | 51 | N/A | 39% |
| formula | 33 | | 49 | | | |
| Boost ® Kid | 17 | 15 | 26 | 20 | 0.1191 | 29% |
| Essentials ™ 1.0 | 15 | | 18 | | | |
| | 13 | | 16 | | | |
| Boost ® Kid | 28 | 25 | 46 | 45 | 0.0006 | 57% |
| Essentials ™ 1.5 | 25 | | 42 | | | |
| | 23 | | 48 | | | |
| Enfamil ® Enspire | 32 | 33 | 39 | 37 | N/A | 13% |
| Optimum ™ | 33 | | 35 | | | |
| Extensive HA ®* | 59 | 57 | 75 | 81 | 0.0065 | 35% |
| | 55 | | 82 | | | |
| | 58 | | 87 | | | |
| Nutramigen ®* | 64 | 64 | 67 | 63 | 0.5483 | −1% |
| | 65 | | 64 | | | |
| | 62 | | 59 | | | |
| Osmolite ® 1.2 | 56 | 60 | 51 | 57 | 0.7290 | −5% |
| | 59 | | 62 | | | |
| | 64 | | 58 | | | |
| Osmolite ® 1.5 | 55 | 50 | 59 | 60 | 0.0356 | 18% |
| | 51 | | 63 | | | |
| | 44 | | 58 | | | |
| PediaSure ® 1.0 | 37 | 36 | 45 | 45 | 0.0009 | 23% |
| | 36 | | 47 | | | |
| | 35 | | 44 | | | |
| Pivot ® 1.5 | 55 | 55 | 77 | 73 | 0.0054 | 28% |
| | 50 | | 73 | | | |
| | 60 | | 68 | | | |
| Similac ® 360 | 34 | 35 | 42 | 44 | N/A | 23% |
| Total Care ® | 34 | | 45 | | | |
| Similac ® | 52 | 49 | 50 | 49 | 0.3773 | 0% |
| Advance ®* | 44 | | 51 | | | |
| | 51 | | 46 | | | |
| Similac ® | 50 | 45 | 47 | 50 | N/A | 11% |
| Alimentum ® | 40 | | 53 | | | |
| Similac ® Pure | 32 | 32 | 39 | 39 | N/A | 18% |
| Bliss ® | 34 | | 38 | | | |

*First pass executed over 30 seconds. All other formulas were tested with a 1 minute first pass.

### EXAMPLE 5

Experiments were performed to compare the hydrolysis activity of lipase on fats in a nutritional formula using a single-pass method and a double-pass method, as disclosed herein. The experiments were performed using Alfamino® infant formula, available from Nestle, EleCare®, available from Abbott Nutrition, Enfamil® EnfaCare® NeuroPro®, available from Mead Johnson, Enfamil® ProSobee®, available from Mead Johnson, Extensive HAR infant formula, available from Nestle, Fortini™, available from Nutricia North America, Neocate® Infant DHA/ARA, available from Nutricia North America, Similac® Advance®, available from Abbott Nutrition, Similac® Alimentum®, available from Abbot Nutrition, and Similac® NeoSure®, available from Abbott Nutrition. These formulas are generally considered harder to hydrolyze using the traditional single-pass method.

To test the single-pass method for Fortini™ and Similac® Advance®, 10 mL of each formula was pushed from a syringe through a hydrolysis device via a syringe pump at a rate of 100 mL/hour. To test the single-pass method for Alfamino® infant, EleCare®, Enfamil® EnfaCare® NeuroPro®, Enfamil® ProSobee®, Extensive HAR infant formula, Neocate® Infant DHA/ARA, Similac® Alimentum®, and Similac® NeoSure®, 50 mL of each formula was pushed from a syringe through a hydrolysis device via a syringe pump at a rate of 100 mL/hour.

To test the double-pass method for Fortini™ and Similac® Advance®, a syringe was attached to a hydrolysis device, and 10 mL of each formula was drawn into the syringe for a total duration of 1 minute, such that the infant formula passed through the hydrolysis device was exposed to the lipase for 1 minute. The infant formula was then pushed from the syringe and back through the hydrolysis device and lipase, via a syringe pump, at a rate of 100 mL/hour.

To test the double-pass method for Alfamino® infant, EleCare®, Enfamil® EnfaCare® NeuroPro®, Enfamil® ProSobee®, Extensive HAR infant formula, Neocate® Infant DHA/ARA, Similac® Alimentum®, and Similac® NeoSure®, a syringe was attached to a hydrolysis device, and 50 mL of each formula was drawn into the syringe at a rate of 100 mL/hour for a total duration of 1 minute, such that the infant formula passed through the hydrolysis device was exposed to the lipase within for 1 minute. The infant formula was then pushed manually from the syringe and back through the hydrolysis device and lipase, via a syringe pump, at a rate of 100 mL/hour.

The results in Table 5 show the percent hydrolysis for each of the individual runs, as well as an average of the runs, for the single pass and double pass. Free fatty acids in the hydrolyzed nutritional formula were measured using a colorimetric assay. Nine of the 10 formulas showed an increase in hydrolysis with use of the double pass method. T-tests were conducted to determine statistical significance (P value <0.05). Six of the 10 formulas had a statistically significant increase in hydrolysis due with use of the double pass method—Alfamino® Infant, EleCare®, Enfamil® EnfaCare® NeuroPro®, Fortini™, Similac® Alimentum®, and Similac@ Advance®.

TABLE 5

| | | | | | | % Difference |
|---|---|---|---|---|---|---|
| | Single Pass | | Double Pass | | P | from Single Pass |
| Formula | % Hydrolysis | | % Hydrolysis | | Value | to Double Pass |
| Alfamino ® | 46 | 45 | 49 | 51 | 0.0044 | 12% |
| Infant | 45 | | 52 | | | |
| | 44 | | 51 | | | |
| EleCare ® | 60 | 58 | 61 | 64 | 0.0321 | 9% |
| | 56 | | 63 | | | |
| | 58 | | 67 | | | |
| Enfamil ® | 54 | 55 | 60 | 59 | 0.0200 | 8% |
| EnfaCare ® | 54 | | 61 | | | |
| NeuroPro ® | 56 | | 57 | | | |
| Enfamil ® | 43 | 42 | 33 | 34 | 0.9797 | −19% |
| ProSobee ® | 42 | | 38 | | | |
| | 40 | | 32 | | | |
| Extensive HA ® | 46 | 52 | 57 | 59 | 0.1059 | 12% |
| | 59 | | 61 | | | |
| | 57 | | 59 | | | |
| Fortini ™ | 61 | 59 | 66 | 66 | 0.0064 | 11% |
| | 59 | | 68 | | | |
| | 57 | | 64 | | | |
| Neocate ® Infant | 41 | 51 | 47 | 53 | 0.3786 | 4% |
| DHA/ARA | 54 | | 56 | | | |
| | 58 | | 56 | | | |
| Similac ® | 60 | 56 | 66 | 65 | 0.0148 | 14% |
| Advance ® | 54 | | 65 | | | |
| | 55 | | 63 | | | |
| Similac ® | 55 | 49 | 68 | 67 | 0.0176 | 31% |
| Alimentum ® | 51 | | 63 | | | |
| | 41 | | 69 | | | |
| Similac ® | 50 | 50 | 50 | 51 | 0.1566 | 3% |
| NeoSure ® | 49 | | 53 | | | |
| | 50 | | 50 | | | |

### EXAMPLE 6

Necrotizing enterocolitis (NEC) is a life-threatening gastrointestinal illness affecting premature infants. Formula feeding is a major risk factor for NEC. The composition of formula, including fat content, impacts the severity of NEC. Premature, formula-fed neonates are lipase-deficient, leading to impaired fat digestion. In this example, the double-pass method was used to hydrolyze fat into more readily absorbable free fatty acid forms, and the effect of an enteral formula hydrolyzed by systems and methods of the present disclosure on NEC mortality and severity was evaluated.

NEC was induced in C57BL/6J mice via oral gavage of formula containing E. coli lipopolysaccharide (LPS E. coli) (2.5 ug/g) and hypoxia exposure (10 min twice daily at 95% nitrogen/5% oxygen concentration). The hydrolysis device used was referred to as an immobilized lipase cartridge (ILC).

Mice were randomly grouped into five treatment groups, including six mice in each group. Group 1 served as the control. In Group 1, mice were kept with the mother for consumption of breast milk for the duration of the study. In Group 2, referred to as the NEC+Placebo group, mice were separated from the mom and placed in a 37° C. incubator. These mice received NEC formula containing LPS E. coli processed through a placebo cartridge using the double-pass method four times daily via gavage and were exposed to hypoxia for 10 minutes twice a day. In Group 3, referred to as the NEC+ILC group, mice were separated from the mom and placed in a 37° C. incubator. These mice received NEC formula containing LPS E. coli processed through the ILC using the double-pass method four times daily via gavage and were exposed to hypoxia for 10 minutes twice a day. In Group 4, referred to as the Formula+Placebo group, mice were separated from the mom and placed in a 37° C. incubator. These mice received infant formula processed through a placebo cartridge using the double-pass method four times daily via gavage. In Group 5, referred to as the Formula+ILC group, mice were separated from the mom and placed in a 37° C. incubator. These mice received infant formula processed through the ILC using the double-pass method four times daily via gavage.

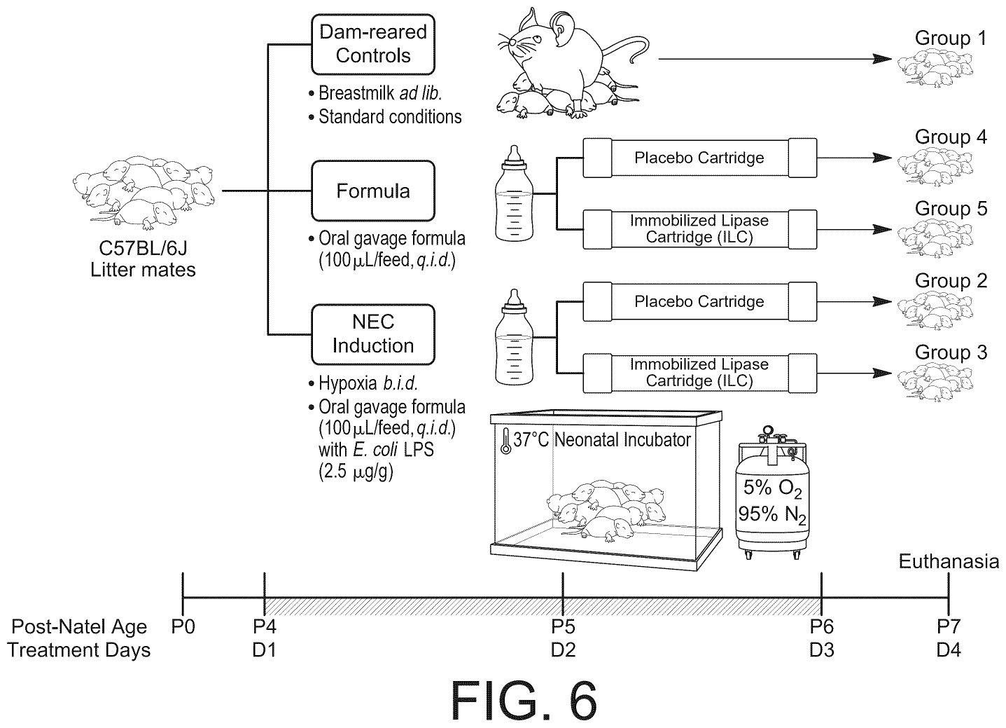

FIG. 6 schematically depicts the study design and procedures for the mice of Groups 1-5.

The formula was prepared by mixing 70 mL of Similac® Advance® OptiGro® with liquid iron and 35 mL Esbilac® Puppy Milk Replacer Liquid. The NEC+ILC (Group 3) and Formula+ILC (Group 5) were prepared according to the double-pass methods of the present disclosure. A 20 mL enteral syringe was connected to an ILC. Ten mL of the formula was drawn through the ILC and into the syringe, such that the infant formula passed through the ILC was exposed to the lipase on a first pass. The syringe and ILC was then assembled into a syringe pump and the infant formula was expelled through the syringe and through the ILC on a second pass at 48 mL/hour. The NEC+Placebo (Group 2) and Formula+Placebo (Group 4) were prepared according to the same protocol as described above, however, the placebo did not include lipase.

The animals were fed up to 80 μL per feed, 4 times per day, over a 12-hour period each day from post-natal day P4 through day P6. Two of the feeding times each day coincided with the 10-minute periods in the hypoxia chamber. The animals were sacrificed on post-natal day P7.

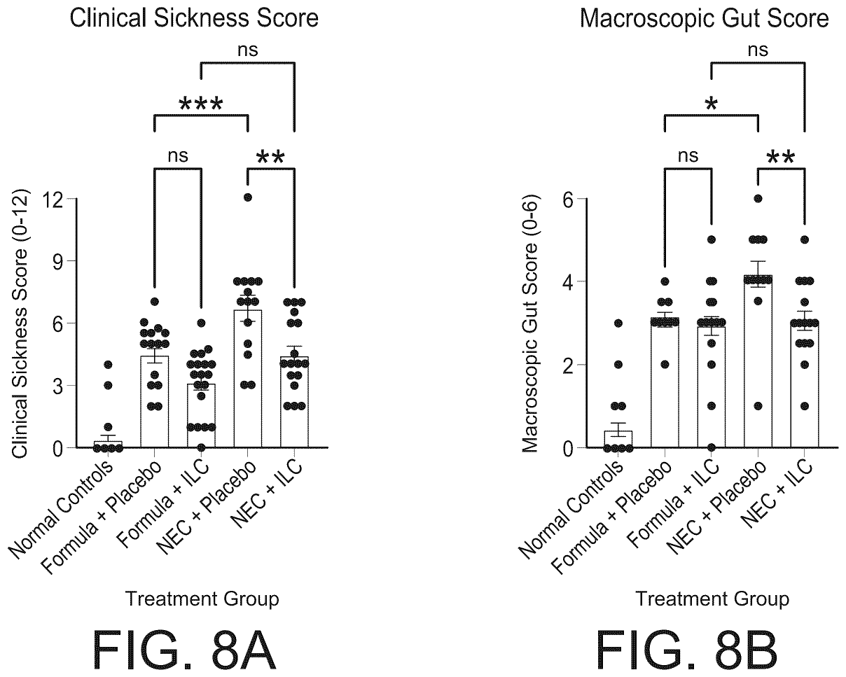

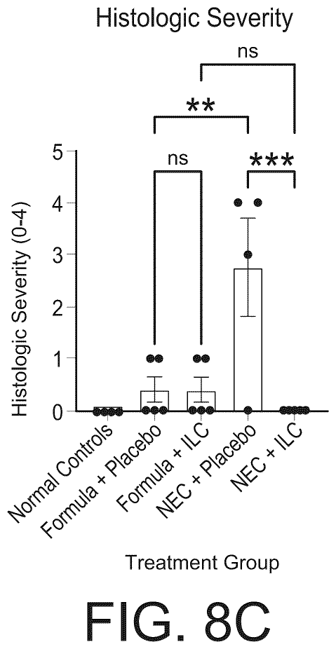

Before euthanasia on post-natal day P7, two masked observers independently assessed clinical sickness score (0-12, increasing severity) (FIG. 8A) and macroscopic gut appearance scores (0.6, increasing severity) (FIG. 8B) according to published protocol, using variables such as response to touch and gut friability. The terminal ileum was resected, formalin-fixed, paraffin-embedded, and stained with hematoxylin and eosin (H&E). A masked veterinary pathologist assessed the histologic severity of NEC according to a published protocol referred to as Camuesco grading (FIG. 8C). NEC severity was graded on a scale from 0 to 4, with 4 being the most severe.

The Shapiro-Wilk test was used to assess data distribution. Outcomes were assessed using analysis of variance (ANOVA), two-sample t-test, or log-rank test where applicable, and described as the mean±the standard error of the mean (SEM) or 95% confidence interval (CI) where appropriate. Tests of significance were two-sided with P<0.05 deemed statistically significant. GraphPad Prism (v10.3.1) software was used for analysis and generation of plots.

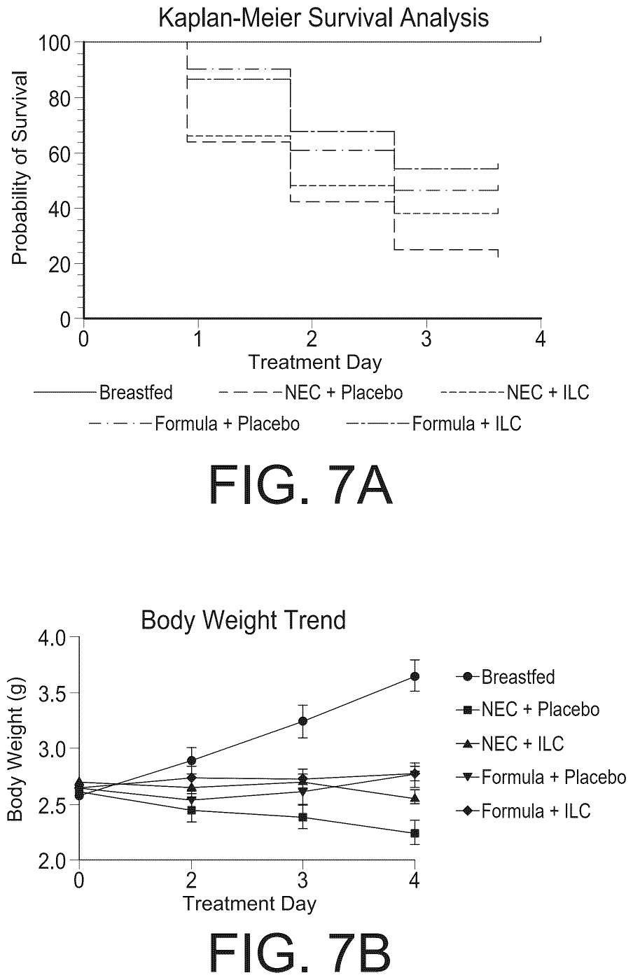

Referring to FIG. 7A, the controls of Group 1 had a 100% survival rate on post-natal day P7. Group 4 Formula+Placebo and Group 5 Formula+ILC had a similar survival rate on log-rank test (46.34% vs 54.05%, P=0.57). Group 2 NEC+Placebo had a similar survival rate to Group 3 NEC+ILC (21.54% vs. 36.54%, P=0.14). All groups had similar starting weights on post-natal day P4 and the weight trends over time are illustrated in FIG. 7B. Group 1 experienced steady weight gain from baseline on post-natal day P4 to the end of the experiment on post-natal day P7 (1.071±0.128 g). Group 5 Formula+ILC weighed 0.09 g greater than Group 4 Formula+Placebo group on post-natal day P6 (95% CI [−0.28, 0.46]), P=0.64), and 0.21 g greater on post-natal day P7 (95% CI [−0.09, 0.52], P=0.17), though these differences did not reach statistical significance. Group 3 NEC+ILC weighed 0.34 g greater than Group 2 NEC+Placebo group on post-natal day P6 (95% CI [0.04, 0.64], P=0.03); however, this same difference did not reach statistical significance on post-natal day P7 (P=0.08). The intraclass correlation for clinical sickness and macroscopic gut scores were 0.91 (95% CI [0.87, 0.94]) and 0.92 (95% CI [0.87, 0.95]) respectively.

Clinical sickness and macroscopic gut scores are presented in FIGS. 8A and 8B, respectively. Referring to FIGS. 8A-8C, ns indicates P>0.05, *P≤0.05, **P≤0.01, ***P≤0.001. Clinical sickness, assessed from 0 to 12 in increasing severity, was lowest in Group 1 at 0.3478±0.21. Group 4 Formula+Placebo had similar scores compared to Group 5 Formula+ILC (4.461±0.31 vs 3.138±0.35, P=0.080). Group 3 NEC+ILC had lower clinical sickness score than Group 2 NEC+Placebo (4.471±0.43 vs 6.714±0.63, P=0.0052). The clinical sickness score of Group 3 NEC+ILC was not statistically different from that of Group 4 Formula+Placebo (P=0.98) and Group 5 Formula+ILC (P=0.21). Macroscopic gut scores, assessed from 0 to 6 in increasing severity, was lowest in Group 1 at 0.4348±0.16. Group 4 Formula+Placebo and Group 5 Formula+ILC had similar gut scores (3.158±0.10 vs 2.925±0.23, P=0.37). Group 3 NEC+ILC had lower gut scores than Group 2 NEC+Placebo (3.059±0.22 vs 4.179±0.30, P=0.0047). Group 3 NEC+ILC additionally had similar gut scores to Group 4 formula+placebo (P=0.67) and Group 5 Formula+ILC (P=0.68).

The histologic severity of NEC was quantified from 0 to 4, in order of increasing severity (FIG. 8C). Control Group 1 had the lowest histologic severity scores with an average of 0.000±0.00. Group 4 Formula+Placebo and Group 5 Formula+ILC had identical average scores of 0.400±0.24. Group 2 NEC+Placebo scored higher than Group 3 NEC+ILC (3.000±0.77 vs 0.000±0.00, P=0.0047) in histologic severity. Group 3 NEC+ILC group had similar histologic severity scores compared to Group 4 Formula+Placebo (P=0.14) and Group 5 Formula+ILC (P=0.14).

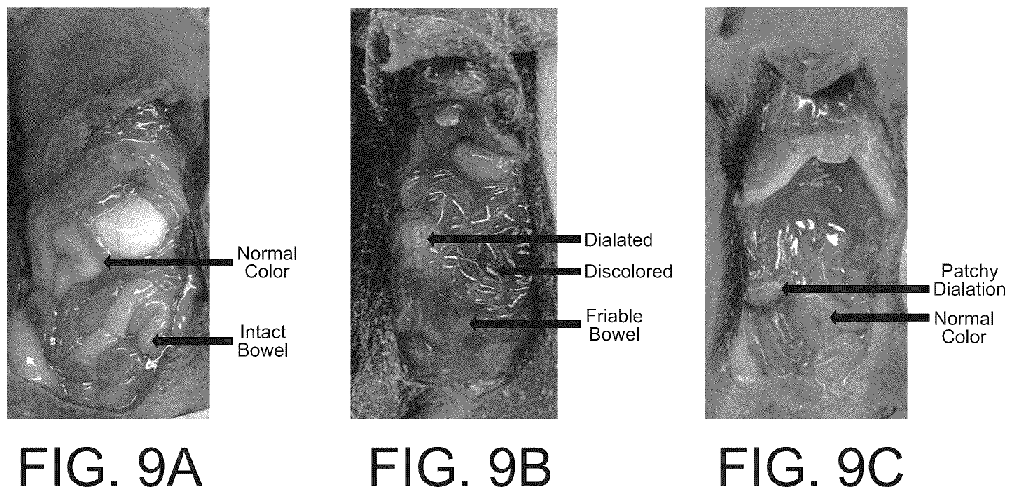

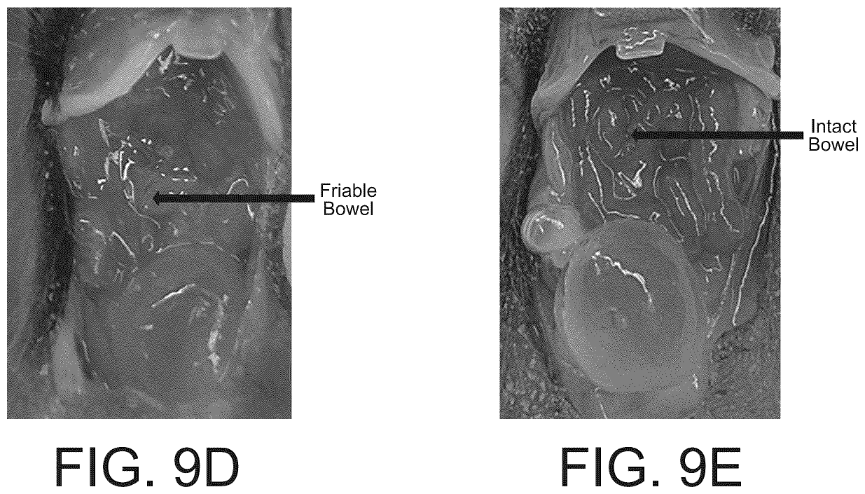

FIGS. 9A-9E are representative photographs of the intestines of the specimens of Group 1 (FIG. 9A), Group 2 NEC+Placebo (FIG. 9B), Group 3 NEC+ILC (FIG. 9C), Group 4 Formula+Placebo (FIG. 9D), and Group 5 Formula+ILC (FIG. 9E). Control Group 1 (FIG. 9A) had normal colored intestines and intact bowels and were remarked as having “no significant findings” per the masked pathologist. Group 2 NEC+Placebo (FIG. 9B) had greater intestinal dilation (abnormal or excessive widening, i.e., expansion, of the intestine), discoloration, villus destruction (e.g., irritation, bleeding or risk of bleeding), and a more friable bowel with multiple dilated segments, compared to Group 3 NEC+ILC (FIG. 9C). Group 4 Formula+Placebo (FIG. 9D) and Group 5 Formula+ILC (FIG. 9E) had intact intestine with rare segmental friability. Groups 4 and 5 also had preserved villi with “minimal inflammation and edema” per the pathologist.

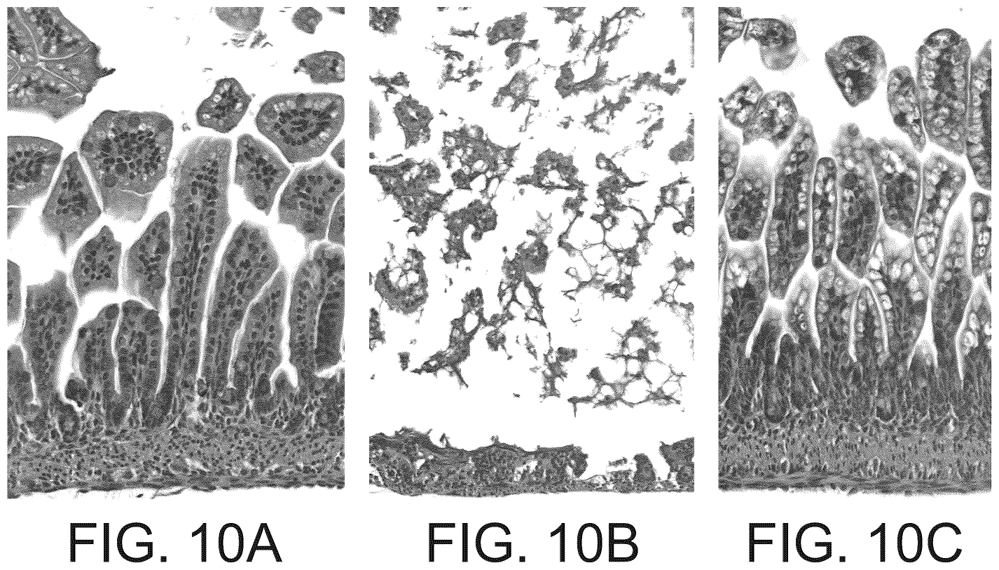

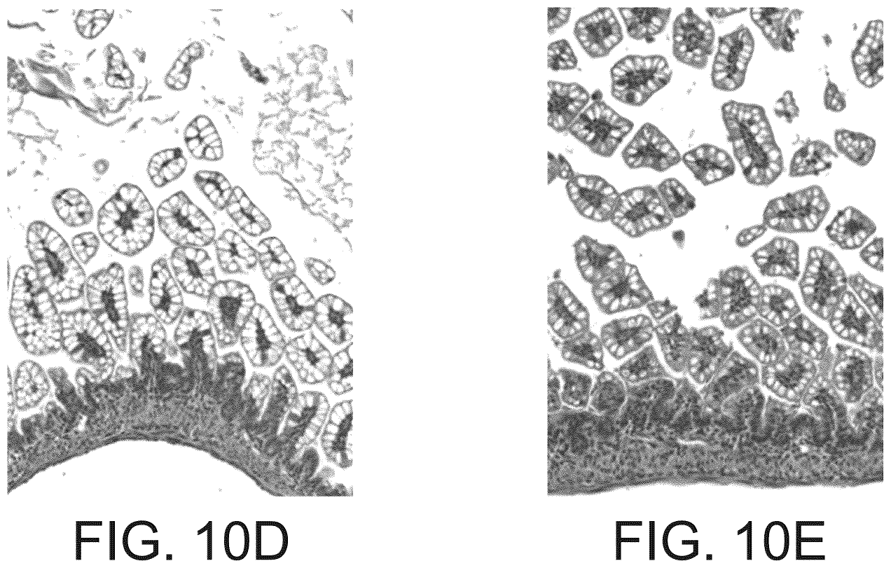

FIGS. 10A-10C depict representative hematoxylin and eosin (H&E) stained terminal ileum (i.e., the last part of the small intestine) specimens of Control Group 1 (FIG. 10A), Group 2 NEC+Placebo (FIG. 10B), Group 3 NEC+ILC (FIG. 10C), Group 4 Formula+Placebo (FIG. 10D), and Group 5 Formula+ILC (FIG. 10E). Control Group 1 had a normal villus architecture. Group 3 NEC+ILC had an intact bowel with patchy areas of dilation, but a preserved intestinal villus architecture and milk inflammation compared to Group 2 NEC+placebo, which had effaced villi with moderate to marked inflammation and extension intestinal erosions, or in other words, complete or almost complete destruction of villi. Group 4 Formula+Placebo and Group 5 Formula+ILC had comparable levels of preserved intestinal villus architecture.

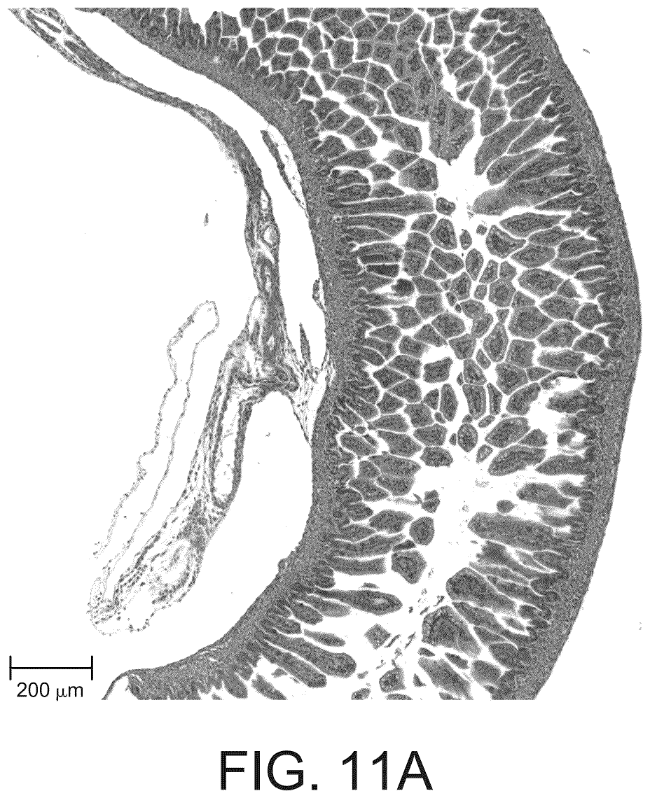

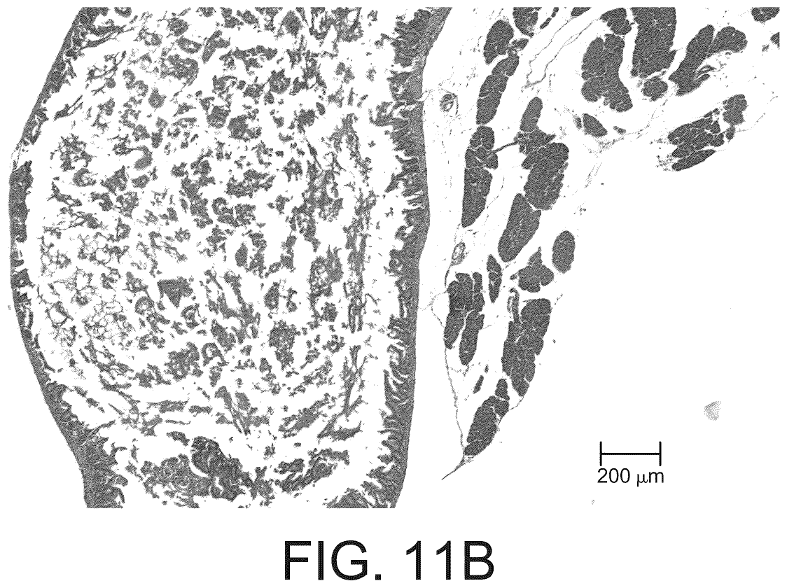

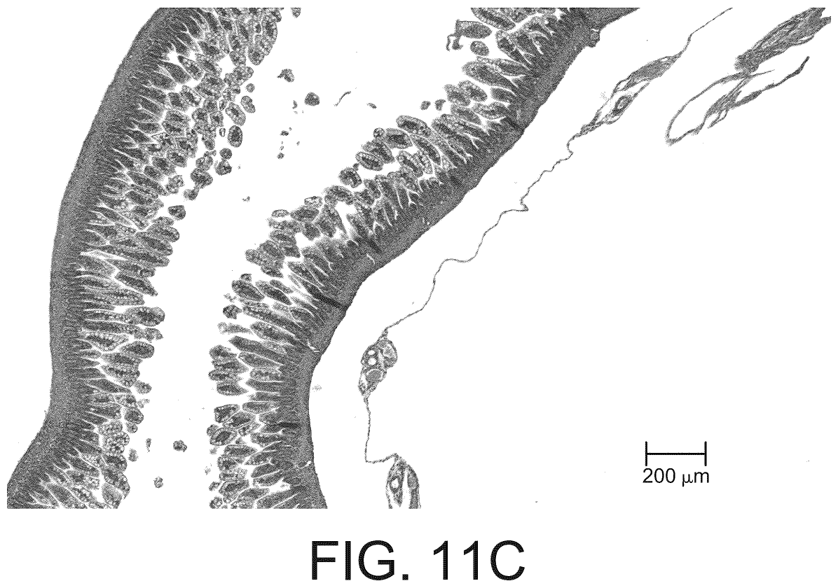

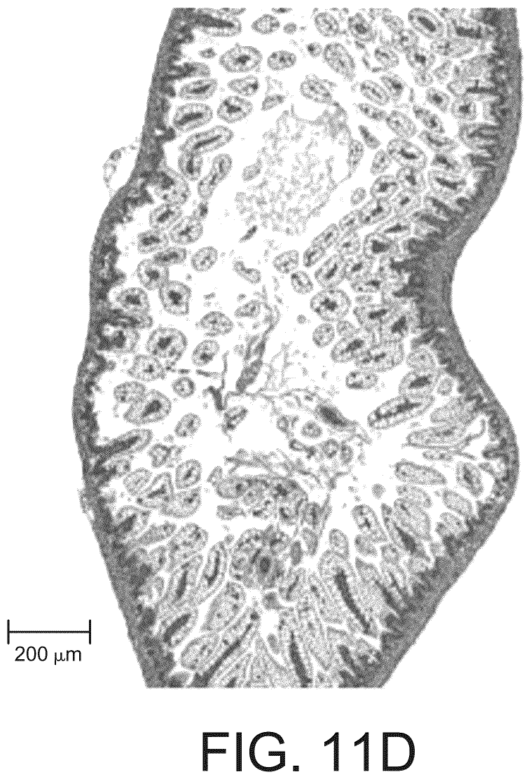

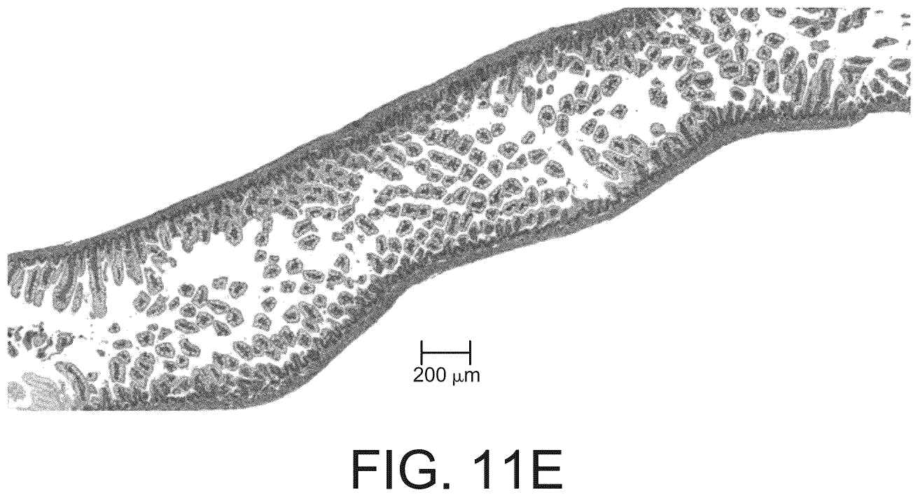

FIGS. 11A-11E depict representative H&E stained villi of the small intestine for Group 1 (FIG. 11A) having intact layers and normal villi, Group 2 (FIG. 11B) having near-perforation, i.e., severe thinning, weakening, and/or damage to the intestinal wall, and destruction of villi and crypts, Group 3 (FIG. 11C) having intact villi and crypts and proliferation of goblet cells (i.e., mucin producing cells in the mucous membranes of the gastrointestinal tracts), Group 4 (FIG. 11D) having abnormal villi and segmental dilation, and Group 5 (FIG. 11E) having fairly normal villi and no dilated segments.

Example 6 indicates that administration of enteral formula according to the systems and methods of the present disclosure improved survival compared to undigested formula in the mice incubated with NEC, as well as the mice without NEC. ILC use improved survivability and weight of the mice, and improved clinical sickness score, macroscopic gut appearance score, and histologic severity in the terminal ileum of the mice.

The many features and advantages of the present disclosure are apparent from the detailed specification, and thus, it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described, and accordingly, all suitable modifications and equivalents may be resorted to, falling within the scope of the present disclosure.

Moreover, those skilled in the art will appreciate that the conception upon which this disclosure is based may readily be used as a basis for designing other structures, methods, and systems for carrying out the several purposes of the present disclosure. Accordingly, the claims are not to be considered as limited by the foregoing description.

What is claimed is:

1. A method of preparing a nutritional composition, the method comprising:
connecting a first end of a hydrolysis device to a syringe, wherein the hydrolysis device contains lipase within a chamber of the hydrolysis device;
drawing a nutritional composition into a second end of the hydrolysis device so that the nutritional composition flows through the hydrolysis device in a first direction and into the syringe, wherein the nutritional composition includes triglycerides; and
expelling the nutritional composition from the syringe, so that the nutritional composition flows through the hydrolysis device in a second direction, opposite the first direction, wherein the drawing and the expelling steps result in a hydrolyzed nutritional composition in which at least some of the triglycerides are hydrolyzed into free fatty acids and monoglycerides.

2. The method of claim 1, wherein the nutritional composition flows through the hydrolysis device in the first direction for a time duration ranging from about 1 second to about 5 minutes.

3. The method of claim 1, wherein the nutritional composition is one of an enteral formula, an infant formula, or milk.

4. The method of claim 1, wherein the lipase contained within the hydrolysis device is immobilized.

5. The method of claim 1, further comprising delivering the hydrolyzed nutritional composition to a subject.

6. The method of claim 1, further comprising connecting an enteral straw to a second end of the hydrolysis device prior to drawing the nutritional composition into the second end of the hydrolysis device so that the nutritional composition flows into the enteral straw and then flows through the hydrolysis device in the first direction.

7. The method of claim 1, further comprising connecting an enteral straw to a second end of the hydrolysis device prior to drawing the nutritional composition into the second end of the hydrolysis device so that the nutritional composition flows into the enteral straw and then flows through the hydrolysis device in the first direction;
removing the enteral straw from the second end of the hydrolysis device prior to expelling the nutritional composition from the syringe; and
connecting a feeding tube to the second end of the hydrolysis device prior to expelling the nutritional composition from the syringe.

8. The method of claim 1, further comprising connecting a feeding tube to the second end of the hydrolysis device prior to expelling the nutritional composition from the syringe.

9. The method of claim 8, further comprising delivering the hydrolyzed nutritional composition to a subject via the feeding tube.

10. The method of claim 1, wherein expelling the nutritional composition from the syringe comprises using a syringe pump.

11. The method of claim 1, further comprising delivering the hydrolyzed nutritional composition to a subject diagnosed with necrotizing enterocolitis.

12. A method of preparing a nutritional composition, the method comprising:
connecting a first end of a hydrolysis device to a syringe, wherein the hydrolysis device contains immobilized lipase within a chamber of the device;
pulling back a plunger of the syringe to draw a nutritional composition through the hydrolysis device a first time and into the syringe, wherein the nutritional composition comprises triglycerides, and wherein at least some of the triglycerides in the nutritional composition are hydrolyzed when exposed to the immobilized lipase within the hydrolysis device the first time;
pushing the plunger of the syringe to expel the nutritional composition out of the syringe and through the hydrolysis device a second time, wherein at least some of the triglycerides in the nutritional composition are hydrolyzed when exposed to the immobilized lipase within the hydrolysis device the second time.

13. The method of claim 12, further comprising connecting an enteral straw to a second end of the hydrolysis device prior to pulling back the plunger of the syringe, so that pulling back the plunger of the syringe causes the nutritional composition to flow into the enteral straw prior to flowing through the hydrolysis device the first time.

14. The method of claim 12, further comprising:
connecting an enteral straw to a second end of the hydrolysis device prior to pulling back the plunger of the syringe, so that pulling back the plunger of the syringe causes the nutritional composition to flow into the enteral straw prior to flowing through the hydrolysis device the first time; and
removing the enteral straw from the second end of the hydrolysis device prior to pushing the plunger of the syringe to expel the nutritional composition out of the syringe.

15. The method of claim 12, further comprising:
connecting an enteral straw to a second end of the hydrolysis device prior to pulling back the plunger of the syringe, so that pulling back the plunger of the syringe causes the nutritional composition to flow into the enteral straw prior to flowing through the hydrolysis device the first time;
removing the enteral straw from the second end of the hydrolysis device prior to pushing the plunger of the syringe to expel the nutritional composition out of the syringe; and
connecting a feeding tube to the second end of the hydrolysis device prior to pushing the plunger of the syringe to expel the nutritional composition out of the syringe.

16. The method of claim 12, further comprising connecting a feeding tube to the second end of the hydrolysis device prior to pushing the plunger of the syringe to expel the nutritional composition out of the syringe.

17. The method of claim 12, wherein pushing the plunger of the syringe to expel the nutritional composition comprises using a syringe pump.

18. The method of claim 12, wherein the nutritional composition is one of an enteral formula, an infant formula, or milk.

19. The method of claim 12, wherein the nutritional composition flows through the hydrolysis device for the first time for a time duration ranging from about 1 second to about 5 minutes.

20. The method of claim 12, further comprising delivering the hydrolyzed nutritional composition to a subject diagnosed with necrotizing enterocolitis.

21. A method of preparing a nutritional composition, the method comprising:
connecting a first end of a hydrolysis device to a syringe, wherein the hydrolysis device contains lipase within a chamber of the device;
pulling back a plunger of the syringe to draw a nutritional composition through the hydrolysis device a first time and into the syringe, wherein the nutritional composition flows through the hydrolysis device the first time for a time duration ranging from about one second to about five minutes; and
using a syringe pump to expel the nutritional composition out of the syringe and through the hydrolysis device a second time, wherein triglycerides in the nutritional composition are hydrolyzed when exposed to the lipase within the device the first time and the second time.

22. The method of claim 21, wherein the nutritional composition is one of an enteral formula, an infant formula, or milk.

23. The method of claim 21, wherein the lipase contained within the hydrolysis device is immobilized.

24. The method of claim 21, further comprising connecting an enteral straw to a second end of the hydrolysis device prior to pulling back the plunger of the syringe, so that pulling back the plunger of the syringe causes the nutritional composition to flow into the enteral straw prior to flowing through the hydrolysis device the first time.

25. The method of claim 21, further comprising:
connecting an enteral straw to a second end of the hydrolysis device prior to pulling back the plunger of the syringe, so that pulling back the plunger of the syringe causes the nutritional composition to flow into the enteral straw prior to flowing through the hydrolysis device the first time;
removing the enteral straw from the second end of the hydrolysis device prior to using the syringe pump to expel the nutritional composition out of the syringe.

26. The method of claim 21, further comprising:
connecting an enteral straw to a second end of the hydrolysis device prior to pulling back the plunger of the syringe, so that pulling back the plunger of the syringe causes the nutritional composition to flow into the enteral straw prior to flowing through the hydrolysis device the first time;
removing the enteral straw from the second end of the hydrolysis device prior to using the syringe pump to expel the nutritional composition out of the syringe; and
connecting a feeding tube to the second end of the hydrolysis device prior to using the syringe pump to expel the nutritional composition out of the syringe.

27. The method of claim 21, further comprising connecting a feeding tube to the second end of the hydrolysis device prior to using the syringe pump to expel the nutritional composition out of the syringe.

28. The method of claim 27, further comprising delivering the hydrolyzed nutritional composition to a subject via the feeding tube.

29. The method of claim 21, further comprising delivering the hydrolyzed nutritional composition to a subject diagnosed with necrotizing enterocolitis.
